(19) United States
(12) Patent Application Publication
(10) Pub. No.: US 2021/0172032 A1
(43) Pub. Date: Jun. 10, 2021

(54) PROCESS FOR SUGAR PRODUCTION

(71) Applicant: NUTRITION SCIENCE DESIGN PTE. LTD, Singapore (SG)

(72) Inventor: David KANNAR, Singapore (SG)

(21) Appl. No.: 17/263,759

(22) PCT Filed: Jul. 30, 2019
(86) PCT No.: PCT/SG2019/050377

§ 371 Date: Jan. 27, 2021

(30) Foreign Application Priority Data
Jul. 30, 2018 (SG) 10201806479U
Aug. 23, 2018 (SG) 10201807139Q

(51) Int. Cl. C13B 50/00; G01N 33/14; C13B 10/02; G01N 21/359

(52) U.S. Cl. CPC C13B 50/00; G01N 21/359; C13B 10/02; G01N 33/143

(57) ABSTRACT

The application relates to a method for producing a sugar product and a system thereof comprising receiving a first input in a control system representative of a pre-treatment sugar composition characteristic and/or an additive; receiving a second input representative of a post-treatment sugar product target specification; using the control system to determine at least one operating parameter for addition of the additive to the pre-treatment sugar and adding the additive based on the at least one determined operating parameter, wherein the at least one determined operating parameter is determined from at least: the first input, the second input, and a correlation relating at least the first input and the second input to the at least one operating parameter; and treating the pre-treatment sugar composition by addition of the additive to produce a post-treatment sugar product with a characteristic that is at or nearer to the target specification than the characteristic of the pre-treatment sugar composition. The system includes at least one spray system for addition of an additive to a pre-treatment sugar composition; at least one sensor for determining one or both of a pre-treatment sugar composition characteristic, or an additive characteristic, and a post-treatment sugar product characteristic and a control system configured to operate as mentioned above.

### FIELD OF THE INVENTION

The invention relates to sugar compositions and improved methods of producing a sugar product. In particular, the present invention relates to sugar with a low glycaemic response (GR), low glycaemic index (GI) and/or low glycaemic load (GL) and processes for their preparation.

### BACKGROUND OF THE INVENTION

Sugar is presented in many different forms from unrefined panela to refined white sugar. Refining to a 99.9 wt % white sugar effectively removes all vitamins, minerals and phytochemical compounds leaving a “hollow nutrient”. Retention of vitamins, minerals and phytochemicals in sugar has been demonstrated to improve health and lower glycaemic index (GI). There is a need for improved low GI and/or low GL sugar compositions.

However, low glycaemic sugar is not commonly used in industry in the preparation of foods containing sugar. The vast majority of the sugar used as an ingredient in industry is refined white sugar. The use of low glycaemic raw sugar by the food industry is likely to increase if sugar of that type could be produced at lower cost and/or with low hygroscopicity. In addition, variations of cost effective processes are needed to allow for production of low glycaemic sugars from sources with difference characteristics and to allow for production of suitable sugar at either a primary sugar mill or a sugar refinery.

There is a need for low glycaemic sugars produced with lower cost and/or greater specificity and/or reproducibility.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

In a first aspect of the invention, there is provided a method for producing a sugar product including:
receiving a first input in a control system representative of a pre-treatment sugar composition characteristic or an additive composition characteristic;
receiving a second input in the control system representative of a post-treatment sugar product target specification;
using the control system to determine at least one operating parameter for addition of an additive to the pre-treatment sugar and operating the addition of the additive in accordance with the at least one determined operating parameter, wherein the at least one determined operating parameter is determined from at least:
   the first input,
   the second input, and
   a correlation relating at least the first input and the second input to the at least one operating parameter; and
treating the pre-treatment sugar composition by addition of the additive to produce a post-treatment sugar product with a characteristic that is at or nearer to the target specification than the characteristic of the pre-treatment sugar composition. In one embodiment of the first aspect of the invention, the first input is representative of a pre-treatment sugar composition characteristic. Alternatively, the first input is representative of an additive composition characteristic.

In a second aspect of the invention, there is provided a method for producing a sugar product including:
receiving a first input in a control system representative of a pre-treatment sugar composition characteristic;
receiving a second input in the control system representative of a post-treatment sugar product target specification;
receiving a third input in the control system representative of an additive composition characteristic;
using the control system to determine at least one operating parameter for addition of an additive to the pre-treatment sugar and operating the addition of the additive in accordance with the at least one determined operating parameter, wherein the at least one determined operating parameter is determined from two or more of the inputs selected from:
   the first input,
   the second input,
   the third input, and
   a correlation relating at least two or more of the inputs selected from the first input, the second input and the third input to the at least one operating parameter; and
treating the pre-treatment sugar composition by addition of the additive to produce a post-treatment sugar product with a characteristic that is at or nearer to the target specification than the characteristic of the pre-treatment sugar composition.

In one embodiment of the second aspect of the invention, the at least one determined operating parameter is determined from all three of the inputs by a correlation relating all three of the inputs to the at least one operating parameter.

In embodiments of the first, second and fourth to eighth aspects of the invention, the method further comprises receiving a first output in the control system representative of a post-treatment sugar product characteristic.

In embodiments of the first to eighth aspects of the invention disclosed, the method is a method of preparing a sucrose sugar or the system is a system for producing a sucrose sugar. Alternatively, or in addition, the method is for adjusting the specification of a sugar prepared from sugar cane or sugar beet or a system for adjusting the specification of a sugar prepared from sugar cane or sugar beet.

In embodiments of the first to eighth aspects of the invention disclosed, the pre-treatment sugar composition is a sugar composition that has been washed, for example by centrifugal washing, but is prior to treatment by addition of an additive. A range of different pre-treatment sugar compositions can be treated according to the above method. The pre-treatment sugar composition can be a refined sugar (ie with no polyphenol content), an unrefined sugar, a raw sugar, or the like. In many examples, the pre-treatment sugar is a sugar that has been processed from massecuite by washing. In many examples, the pre-treatment sugar is crystalline. In some embodiments, the pre-treatment sugar is a refined white crystalline sugar. This sugar may be either refined cane sugar or refined beet sugar. In other embodiments, the pre-treatment sugar is has polyphenol content, for example, because the pre-treatment was only partially washed from cane sugar massecuite such that polyphenol content remains in the pre-treatment sugar. In some embodiments, the pre-treatment sugar comprises less than 40 mg CE polyphenols/100 g carbohydrates. Alternatively, the pre-treatment sugar comprises 5 to 40 mg CE polyphenols/100 g carbohydrates, 5 to 30 mg CE polyphenols/100 g carbohydrates or 5 to 20 mg CE polyphenols/100 g carbohydrates. Preferably, the polyphenols include tricin, luteolin and/or apigenin. Optionally, where the post treatment sugar has 46 to 100 mg CE polyphenols/100 g carbohydrates, the pre-treatment sugar can have more than 40 mg CE polyphenols/100 g carbohydrates, for example, 41 to 80, 46 to 60 or 41 to 50 mg CE polyphenols/100 g carbohydrates. Therefore, the pre-treatment sugar can be from 5 to 80 or 5 to 50 mg CE polyphenols/100 g carbohydrates. Optionally, the pre-treatment sugar is crystalline and has polyphenols entrained within the sugar crystals.

In embodiments where the pre-treatment sugar comprises polyphenol content, the pre-treatment sugar optionally has about 50% to 95% of the polyphenols on the outside of the sugar particles and about 5% to 50% of the polyphenols within the sucrose crystals. Alternatively, about 60% to 85% of the polyphenols are on the outside of the sugar particles and about 15% to 40% of the polyphenols are within the sucrose crystals, about 65% to 80% of the polyphenols are on the outside of the sugar particles and about 20% to 45% of the polyphenols are the sucrose crystals. In particular, about 70% to 75% of the polyphenols are on the outside of the sugar particles and about 25% to 30% of the polyphenols are within the sucrose crystals.

In an embodiment of the first to eighth aspects of the invention disclosed, the additive is a liquid. In an alternative embodiment, the additive is a solid. When the additive is a solid, the additive may be a powder or particles. Solid additives may be crystalline or amorphous. When a liquid, the additive may include suspended particles and/or be an emulsion. In some embodiments, the additive includes one or more of polyphenols, protein, fibre, indigestible starch. In some embodiments the additive is an amorphous sugar as described in patent application number SG 10201800837U. In alternate embodiments, the additive is liquid cane juice or a liquid waste stream produced during preparation of the sugar optionally with an increased content of polyphenols, protein, fibre, indigestible starch or a mixture thereof. In preferred embodiments, the additive is at most about 50% w/w carbohydrates. Optionally, the additive is about 30 to about 50% w/w carbohydrates. Optionally, the additive is derived from either cane or beet sugar. Alternatively, the additive is less than 30% carbohydrates or about 5 to about 30% w/w carbohydrates. Optionally, the additive has no carbohydrates. The additive is optionally 500 to 10,000 mg GAE/100 g carbohydrates, 1,000 to 10,000 mg GAE/100 g carbohydrates, 5,000 to 10,000 mg GAE/100 g carbohydrates. Additives of this type can be obtained from cane sugar waste streams and optionally concentrated and/or dried.

The term post-treatment sugar product refers to the sugar product that is arrived at after addition of the additive to the pre-treatment sugar. In embodiments of the first to eighth aspects of the invention disclosed, the post-treatment sugar product comprises sucrose crystals, reducing sugars and polyphenols, wherein the sugar particles comprise about 0 to 0.5 g/100 g reducing sugars and about 20 mg CE polyphenols/100 g carbohydrates to about 45 mg CE polyphenols/100 g carbohydrates and the sugar particles are low glycaemic, ie have a glucose based glycaemic index of less than 55, and/or 10 g of the post-treatment sugar has a glycaemic load (GL) of 10 or less. Alternatively, the post-treatment sugar comprises sucrose crystals, reducing sugars and polyphenols, wherein the sugar particles comprise about 0 to 0.5 g/100 g reducing sugars and about 20 mg CE polyphenols/100 g carbohydrates to about 45 mg CE polyphenols/100 g carbohydrates and wherein a first proportion of the polyphenols are entrained within the sucrose crystals and a second proportion of the polyphenols is distributed on the surfaces of the sucrose crystals and the sugar particles have a glucose based glycaemic index of less than 55 and/or 10 g of the post-treatment sugar has a glycaemic load (GL) of 10 or less. Preferably, the post-treatment sugar product is of food grade quality. Preferably, the polyphenols include tricin, luteolin and/or apigenin.

In embodiments of the first to eighth aspects of the invention disclosed, the post-treatment sugar is a very low glycaemic sugar, optionally comprising at least 80% sucrose. Preferably, the very low glycaemic sugar comprises about 60 mg CE polyphenols/100 g carbohydrates or about 50 mg GAE polyphenols/100 g carbohydrates and is optionally at least 90% or 95% by weight sucrose.

In some embodiments, the post-treatment sugar is low glycaemic and comprises at least about 80% w/w sucrose and about 46 mg CE polyphenols/100 g carbohydrates to about 100 mg CE polyphenols/100 g carbohydrates or about 37 mg GAE polyphenols/100 g carbohydrates to about 80 mg GAE polyphenols/100 g carbohydrates. The post-treatment sugar is optionally 0 to 1.5% w/w reducing sugars, not more than 0.5% w/w fructose and not more than 1% w/w glucose.

In some embodiments, the post-treatment sugar is low glycaemic and comprises about 46 mg CE polyphenols/100 g carbohydrates to about 100 mg CE polyphenols/100 g carbohydrates or about 37 mg GAE polyphenols/100 g carbohydrates to about 80 mg GAE polyphenols/100 g carbohydrates and 0 to 1.5% w/w reducing sugars.

In some embodiments, the post-treatment sugar has a GL of 10 or less, 8 or less, or 5 or less. Calculation of glycaemic load of an amount of a food is explained in the detailed description below. Optionally, the post-treatment sugar has a glucose based GI of 54 or less (ie low glycaemic) or 50 or less. Optionally, the post-treatment sugar has a glucose based GI of 54 or less and 10 g of the post-treatment sugar has a glucose based GL of 10 or less.

In some embodiments, one or both of the pre-treatment sugar and the post-treatment sugar have moisture content of 0.02% to 0.6%, 0.02 to 0.3% 0.02% to 0.2%, 0.1% to 0.5%, 0.1% to 0.4%, 0.1 to 0.2%, 0.2% to 0.3% or 0.3 to 0.4% w/w. Optionally, the post-treatment sugar has 0.02% to 1%, 0.02% to 0.8%, 0.02% to 0.6%, 0.1% to 0.5%, 0.1% to 0.4% or 0.2% to 0.3% w/w moisture content after 6 months storage at room temperature and 40% relative humidity or, alternatively, after 12 months storage at room temperature and 40% relative humidity.

In some embodiments, the pre-treatment and/or post-treatment sugars will fall within the maximum residue limits for chemicals set out in Schedule 20 of the Australian Food Standards Code in force July 2017. Optionally, the sugar particles meet the following pesticide/herbicide levels: less than 5 mg/kg 2,4-dichlorophenoxyacetic acid, less than 0.05 mg/kg paraquat, less than 0.05 mg/kg ametryn, less than 0.1 mg/kg atrazine, less than 0.02 mg/kg diuron, less than 0.1 mg/kg hexazinone, less than 0.02 mg/kg tebuthiuron, less than 0.03 mg/kg glyphosate, a combination of these or all of these.

Alternatively, the pre-treatment and/or post-treatment sugars fall within the following pesticide/herbicide levels: less than 0.005 mg/kg 2,4-dichlorophenoxyacetic acid, less than 0.01 mg/kg diquat, less than 0.01 mg/kg paraquat, less than 0.01 mg/kg ametryn, less than 0.01 mg/kg atrazine, less than 0.05 mg/kg bromacil, less than 0.01 mg/kg diuron, less than 0.05 mg/kg hexazinone, less than 0.01 mg/kg simazine, less than 0.01 mg/kg tebuthiuron, less than 0.01 mg/kg glyphosate, a combination of these or all of these.

An input representative of a pre-treatment sugar composition characteristic, an additive characteristic and/or a target specification, as referred to in the first to eighth aspects of the invention, can be received from a variety of sources; e.g. it can be received from one or more sensors, it can be received by data transfer from another system e.g. a remote system storing or processing operational data such as specification, sensor data or the like, it may be entered into the control system or by a user via a user interface or input device associated with the control system. A combination of sources may be used in a single embodiment.

The method in the first to eighth aspects of the invention advantageously allow the production of a post-treatment sugar product with characteristics that are closer to the target specification than the pre-treatment sugar composition. This is achieved, in part, through the use of a database that includes historical information regarding characterisation of previously processed pre-treatment sugar compositions and/or characterisation of previously used additives to post-treatment sugar products, and the manner of processing. Information regarding characterisation of the pre-treatment sugar composition and/or the additive and a target specification of the post-treatment sugar composition is fed to the control system; the control system considers this information in combination with the historical information to determine an appropriate operating strategy for addition of the additive to produce a sugar product that ideally has the target specification. Alternatively, the control system could refer to an algorithm developed from the historical information to determine an appropriate operating strategy for addition of the additive to produce a sugar product that ideally has the target specification. Thus, in a preferred form, the correlation is derived from a database of historical first inputs, second inputs and/or third inputs and corresponding historical output characterisation data and associated operating parameter(s).

In embodiments including a database of historical information, after the step of subjecting the pre-treatment sugar composition to the addition of the additive, the process further includes: obtaining corresponding output characterisation data from the post-treatment sugar product; and updating the database with the first input, the corresponding output characterisation data, and the operating parameter(s).

It will be appreciated, that due to the wide possible variances in the nature of the pre-treatment sugar compositions and additives, there can be some degree of deviation in the post-treatment sugar product characteristics from the targeted specification. To this end, in an embodiment of each of the first to eight aspects of the invention, after the step of adding the additive to the pre-treatment sugar composition, the process further includes obtaining corresponding output characterisation data from the post-treatment sugar product, and updating the database with the first input and/or third input, the corresponding output characterisation data, and the operating parameter(s) used in processing. In this way, the control system is a closed loop control system that is able to apply heuristics to improve future process control and narrow the variance in the post-treatment sugar products from the target specification.

It will be appreciated that the characteristic or specification may be defined in terms of any measurable physicochemical property of the sugar. For example, the property may be viscosity; hygroscopicity; moisture levels; types and/or concentrations of phytochemicals such as tannins, caramels, flavonoids, mono- and/or polyphenols, and/or reducing sugars; and/or conductivity. The initial characteristic of the pre-treatment sugar and/or the output characterisation may be obtained by determining an ICUMSA rating, measuring conductivity, and/or conducting spectral analysis. Similarly, the target specification may be provided as an ICUMSA rating, conductivity value, and/or spectrum. Generally, it is preferred that the target specification is provided in corresponding form to the initial characterisation, for example, if the initial characterisation is measured as a spectrum, then the target specification may also be provided in the form of a spectrum. Notwithstanding this, the target specification could be provided in terms of a different physicochemical property to the characteristics measured in the pre and/or post treatment sugar products and/or additive and a correlation between the specification domain and characterisation domain used to determine system control parameters. Thus in one or more embodiments, the database includes information regarding sugar compositions and products in the form of an R² value that correlates two sugar properties (ie two inputs selected from the first input, second input and third input). In alternative embodiments, where three or more inputs are being correlated, the database includes information in the form of a multiple correlation coefficient. The R² value or multiple correlation coefficient allows the control system to predict or determine at least one operating parameter for the addition of the additive to target one of those sugar properties based on a pre-treatment sugar characteristic that is the other of those two sugar properties. In one example, the pre-treatment sugar composition characteristic is an NIR spectrum, and the target specification is an ICUMSA value. In this example, the database includes a correlation of NIR spectra data with ICUMSA values, and an appropriate operating parameter for the addition of the additive is then selected using this correlation.

In another example, the pre-treatment sugar composition characteristic is conductivity or ICUMSA, and the target specification is a further sugar characteristic (which may be any other property). In this example, the database includes a correlation of conductivity or ICUMSA with the sugar characteristic values, and an appropriate operating parameter for the centrifuge is then selected using this correlation. It will also be understood that the post-treatment sugar product characteristic is conductivity or ICUMSA, and the pre-treatment specification is the further sugar characteristic.

In an embodiment of each of the first to the eighth aspects of the invention, the pre-treatment sugar composition characteristic is a pre-treatment spectrum, the additive composition characteristic is an additive spectrum, and the post-treatment sugar product target specification is a post-treatment spectrum. These spectra are preferably selected from the group consisting of a colour spectrum, a near infrared (NIR) spectrum, and/or a UV-vis spectrum. More preferably, the spectra are NIR spectra, and preferably determined using NIR or microNIR units. The use of NIR spectra (such as from NIR or microNIR units) is particularly useful in instances where the pre-treatment sugar composition has high ICUMSA. Typically, optimal colour/UV-vis measurements are restricted to a range of 3 to 10,000 IU. The pre-treatment sugar is by nature variable in its specifications but preferably, 1,000 to 5,000 IU or 2,500 to 3,000 IU. However, NIR allows accurate measurements to be taken when the ICUMSA is above 10,000 ICUMSA Units (IU), such as is generally the case with massecuite. Massecuite is more likely to be a pre-wash sugar than a pre-treatment sugar according to the invention.

It is preferred that each spectrum is indicative of a property selected from the group consisting of: flavonoid types and/or concentrations, phenol types and/or concentrations, polyphenol types and/or concentrations, tannin types and/or concentrations, caramel compound types and/or concentrations, reducing sugar types and/or concentrations, and moisture, pol, grain size, sucrose concentration, reducing sugar concentration, ash content, and grain size. In one form of the invention, the spectra are NIR spectra that are indicative of tricin concentration. The inventor has found that tricin is able to be detected by NIR, and that measurement of tricin provides a better and more direct measurement than broadly measuring polyphenols. Thus, using tricin concentration as the pre-treatment sugar composition characteristic and/or the post-treatment sugar product target specification provides greater control and specificity over the method resulting in a sugar product with a profile that more closely matches the target specification.

In an embodiment of each of the first to the eighth aspect of the invention, the target specification is a spectrum correlated with a post-treatment sugar having about 0 to about 0.5 g/100 g reducing sugars. More preferably, the target specification is about 0.05 g/100 g to about 0.25 g reducing sugars. Most preferably, the target specification is about 0.12 g/100 g to about 0.16 g reducing sugars. Alternatively, the target specification is a spectrum correlated with a post-treatment sugar having about 0 to about 1.5% w/w reducing sugars.

In an embodiment of each of the first to the eighth aspect of the invention, the target specification is a spectrum correlated with a post-treatment sugar having about 15 mg CE polyphenols/100 g carbohydrates to about 45 mg CE polyphenols/100 g carbohydrates (or about 12 mg GAE polyphenols/100 g carbohydrates to about 37 mg GAE polyphenols/100 g carbohydrates). More preferably, the target specification is about 20 mg CE (or about 16 mg GAE) polyphenols/100 g carbohydrates to about 40 mg CE (or about 33 mg GAE) polyphenols/100 g carbohydrates. Most preferably, the target specification is correlated with a post-treatment sugar having about 25 mg CE (or about 20 mg GAE) polyphenols/100 g carbohydrates to about 35 mg CE (or about 28 mg GAE) polyphenols/100 g carbohydrates. Alternatively, the target specification is correlated with a post-treatment sugar having about 20 mg CE polyphenols/100 g carbohydrates to about 45 mg CE polyphenols/100 g carbohydrates. Alternatively, the target specification is a spectrum correlated with a post-treatment sugar having polyphenols of about 46 mg CE polyphenols/100 g carbohydrates to about 100 mg CE polyphenols/100 g carbohydrates or about 37 mg GAE polyphenols/100 g carbohydrates to about 80 mg GAE polyphenols/100 g carbohydrates. Preferably, the target specification is correlated with a post-treatment sugar having about 60 mg CE polyphenols/100 g carbohydrates or about 50 mg GAE polyphenols/100 g carbohydrates.

In an embodiment, the target specification is a spectrum correlated with a post-treatment sugar having a moisture content of 0.02% to 0.6%. Preferably, the moisture content is 0.10 to 0.20%. Most preferably, the moisture content is 0.13 to 0.17%.

In an embodiment, the target specification is a colour of about 500 to 2000 IU. More preferably, the target specification is a colour of about 800 to 1800 IU. Most preferably, the target specification is a colour of about 1150 to 1450 IU.

In an embodiment, the target is an electrical conductivity of 100 to 300 pS/cm.

In an embodiment of the first to eighth aspects of the invention, the characteristic of the post-treatment sugar product is within 20% of the target specification. Preferably the characteristic is within 18% of the target specification. More preferably, the characteristic is within 15% of the target specification. Even more preferably, the characteristic is within 12% of the target specification. Still more preferably, the characteristic is within 10% of the target specification. Most preferably, the characteristic is within 5% of the target specification.

As generally discussed above, the control system determines an operating strategy with a view to producing a post-treatment sugar product that has a profile that is consistent with (or approaches) the target specification. While the operating strategy can be in respect of any parameter associated with addition of the additive, it is preferred that the additive is added by spraying and the operating strategy controls one or more parameters selected from the group consisting of: spray time, volume of sprayed solution, force of spray, feed rate and/or spray nozzle shape, angle, position and/or temperature. Preferred parameters include spray time, and volume of spray solution. In some cases, it should be noted that such control may be applied to a device different to the spray device such that the parameter associated with the operation of the spray is controlled, e.g. a valve upstream of the spray device can be controlled to determine spray speed.

Controlling the process in this manner provides a number of advantages over known sugar production methods. In prior art processes, phytochemicals are typically washed out of cane and beet sugars to produce a white sugar. The reason for this is to achieve consistency and uniformity for organoleptic purposes in food, and also to remove impurities such as herbicide residues, and pesticide residues. Furthermore, in some prior art processes colour, polyphenols, phytochemical complexes are also considered impurities and are thus desirably removed. A white sugar can be treated with molasses or sugarcane extract to coat the white sugar with the phytochemicals and thus produce a low GI white sugar coated in phytochemicals.

The inventor of the present invention previously invented a low GI sugar (see international patent publication WO 2018018090) and method for controlling the centrifugal washing process of sugar production to directly prepare a low GI sugar without the need for a respraying process (see international patent publication WO 2018018089, a copy of which is incorporated by reference). That process did not produce a refined white sugar. The process was for implementation at a primary sugar mill and produced the low GI sugar directly from massecuite. Since development of that invention, the inventor has discovered a market for the production of low GI and/or low GL sugar at a refinery rather than a primary mill and a market for preparation of low GI and/or low GL sugar at primary mills where the massecuite has insufficient quantity of polyphenols to achieve a suitable low GI sugar by the centrifugal washing process alone and an addition of further polyphenols is required to prepare a low GI sugar. The addition of the further polyphenols could occur either at the primary mill or at a sugar refinery.

More generally such a method may allow sugar refineries and/or mills to produce a more consistent product. As an alternative or additionally, such methods may reduce the cost of production in terms of either or both of operating costs (by reducing respraying time and/or use of the additive). The heuristics allow the control system to refine the operating parameter(s) of the equipment for addition of the additive to accommodate and adapt to a wide range of pre-treatment sugar composition inputs.

In some embodiments, the method is conducted at a primary sugar mill. In other embodiments, the method is conducted at a sugar refinery.

In an embodiment, the method further includes providing the first input to the control system representative of the pre-treatment sugar composition characteristic.

In an embodiment, the method further includes providing the second input to the control system representative of the post-treatment sugar product target specification.

In an embodiment, the method further includes providing the third input to the control system representative of the post-treatment sugar product target specification.

In a third aspect of the invention, there is provided a system for producing a sugar product including:
at least one spray system for adding an additive to a pre-treatment sugar composition to produce a post-treatment sugar product;
at least one sensor for determining one or more of a pre-treatment sugar composition characteristic, an additive characteristic and a post-treatment sugar product characteristic;
a control system configured to determine at least one operating parameter for the at least one spray system based on two or more of:
   a pre-treatment sugar composition characteristic,
   an additive composition characteristic, and
   a target specification of the post-treatment sugar product; and
   a correlation relating the at least one operating parameter to two or more of the pre-treatment sugar composition characteristic, the additive composition characteristic and the target specification;
wherein the control system is further configured to operate the at least one spray system in accordance with the operating parameter.

In one embodiment of the third aspect of the invention, the at least one determined operating parameter is determined by correlation of all three of the pre-treatment sugar composition characteristic, the additive composition characteristic and the target specification.

In an embodiment of any of the first to eight aspects of the invention, the control system further includes a database of historical pre-treatment sugar composition characteristics and/or additive characteristics, corresponding historical post-treatment sugar product characteristics, and corresponding operating parameter(s) from the at least one spray system; and wherein the correlation is derived from historical information in the database.

In embodiments of the first to eight aspect of the invention comprising sensors, the at least one sensor is for determining a pre-treatment sugar composition characteristic, or an additive characteristic, and a post-treatment sugar product characteristic (ie because the post-treatment sugar outlet is via the sensor used to sense the pre-treatment sugar or additive input). In an alternative embodiment, the at least one sensor is for determining a pre-treatment sugar composition characteristic, an additive characteristic, and a post-treatment sugar product characteristic (ie because the post-treatment sugar outlet is via the sensor used to sense the pre-treatment sugar and the additive input). Preferably, where the system further includes the database, the at least one sensor is configured to update the database with the pre-treatment sugar composition characteristic and/or additive characteristic, the post-treatment sugar product characteristic, and the operating parameter(s). In the first, second and fourth to eight embodiments the first to third inputs can be received by a sensor as described above and below.

In embodiments of the first to eight aspect of the invention comprising sensors, the control system includes at least two sensors, a first sensor to determine the pre-treatment sugar composition characteristic and/or the additive characteristic, and a second sensor to determine the post-treatment sugar product characteristic. Preferably, where the sensor determines the additive characteristic, the first sensor is located upstream of the spray system, such as adjacent an inlet to the nozzle; and the second sensor is located downstream of the spray system, such as adjacent an outlet of the container in which the sugar is sprayed, such as after the sugar has been dried.

In a fourth aspect of the invention, there is provided a method for producing a sugar product including:
receiving a first input in a control system representative of a pre-wash sugar composition characteristic or an additive composition characteristic;
receiving a second input in the control system representative of a post-treatment sugar product target specification;
using the control system to determine at least one operating parameter for addition of an additive to the pre-treatment sugar and operating the addition of the additive in accordance with the at least one determined operating parameter, wherein the at least one determined operating parameter is determined from at least:
   the first input,
   the second input, and
   a correlation relating at least the first input and the second input to the at least one operating parameter; and
treating the pre-wash sugar composition by washing and then by addition of the additive to produce a post-treatment sugar product with a characteristic that is at or nearer to the target specification than the characteristic of the pre-wash sugar composition. In one embodiment of the fourth aspect of the invention, the first input is representative of a pre-wash sugar composition characteristic. Alternatively, the first input is representative of an additive composition characteristic.

In a fifth aspect of the invention, there is provided a method for producing a sugar product including:
receiving a first input in a control system representative of a pre-wash sugar composition characteristic;
receiving a second input in the control system representative of a post-treatment sugar product target specification;
receiving a third input in a control system representative of an additive composition characteristic;
using the control system to determine at least one operating parameter for addition of an additive to the pre-treatment sugar and operating the addition of the additive in accordance with the at least one determined operating parameter, wherein the at least one determined operating parameter is determined from two or more of the inputs selected from:
   the first input,
   the second input,
   the third input, and
   a correlation relating at least two or more of the inputs selected from the first input, the second input and the third input to the at least one operating parameter; and
treating the pre-wash sugar composition by washing and then addition of the additive to produce a post-treatment sugar product with a characteristic that is at or nearer to the target specification than the characteristic of the pre-wash sugar composition.

In one embodiment of the fifth aspect of the invention, the at least one determined operating parameter is determined from all three of the inputs by a correlation relating all three of the inputs to the at least one operating parameter.

In embodiments of the fourth and fifth aspect of the invention, the pre-wash sugar composition is massecuite or a primary mill sugar with more than the desired reducing sugars (eg above 0.18% w/w reducing sugar), polyphenols, herbicides or pesticides, and/or other impurities.

In a sixth aspect of the invention, there is provided a method for producing a sugar product including:
receiving a first input in a control system representative of a pre-wash sugar composition characteristic or an additive composition characteristic;
receiving a second input in the control system representative of a post-treatment sugar product target specification;
using the control system to determine at least one wash operating parameter for washing the pre-wash sugar in a centrifuge and at least one additive operating parameter for addition of an additive to the pre-treatment sugar and operating wash of the sugar in the centrifuge in accordance with the at least one determine wash operating parameter and operating the addition of the additive in accordance with the at least one determined additive operating parameter, wherein both the at least one determined wash operating parameter and the at least one determined additive operating parameter are determined from at least:
   the first input,
   the second input, and
   a correlation relating at least the first input and the second input to the at least one wash operating parameter and the at least one additive operating parameter; and
treating the pre-wash sugar composition by washing and then by addition of the additive to produce a post-treatment sugar product with a characteristic that is at or nearer to the target specification than the characteristic of the pre-wash sugar composition. In one embodiment, the first input is representative of a pre-wash sugar composition characteristic. Alternatively, the first input is representative of an additive composition characteristic.

In a seventh aspect of the invention, there is provided a method for producing a sugar product including:
receiving a first input in a control system representative of a pre-wash sugar composition characteristic;
receiving a second input in the control system representative of a post-treatment sugar product target specification;
receiving a third input in a control system representative of an additive composition characteristic;
using the control system to determine at least one wash operating parameter for washing the pre-wash sugar in a centrifuge and at least one additive operating parameter for addition of an additive to the pre-treatment sugar and operating wash of the sugar in the centrifuge in accordance with the at least one determine wash operating parameter and operating the addition of the additive in accordance with the at least one determined additive operating parameter, wherein both the at least one determined wash operating parameter and the at least one determined additive operating parameter are determined from at least:
   the first input,
   the second input,
   the third input, and
   a correlation relating at least the first input, the second input and the third input to the at least one wash operating parameter and the at least one additive operating parameter; and
treating the pre-wash sugar composition by washing and then by addition of the additive to produce a post-treatment sugar product with a characteristic that is at or nearer to the target specification than the characteristic of the pre-wash sugar composition. Optionally, the first input is representative of a pre-wash sugar composition characteristic. Alternatively, the first input is representative of an additive composition characteristic.

The pre-wash sugar is optionally massecuite. Alternatively, the pre-wash sugar has been washed previously but will be subjected to another wash before addition of the additive to prepare the final sugar product.

The invention also provides for the use of a controlled centrifugal washing process as described in international patent application number PCT/AU2017/050781 followed by the controlled addition of an additive as described herein. For example, in an eighth aspect the invention provides a method for producing a sugar product including:
receiving an alpha input in a control system representative of a pre-wash sugar composition characteristic;
receiving a beta input in the control system representative of a pre-treatment sugar product target specification;
using the control system to determine at least one operating parameter for a centrifuge and operating the centrifuge in accordance with the at least one determined operating parameter, wherein the at least one determined operating parameter is determined from at least:
   the alpha input,
   the beta input, and
   a correlation relating at least the alpha input and the beta input to the at least one operating parameter; and
   treating the pre-wash sugar composition in the centrifuge to produce a pre-treatment sugar product with a characteristic that is at or nearer to the target specification than the characteristic of the pre-wash sugar composition;
receiving a first input in a control system representative of a pre-treatment sugar composition characteristic or an additive composition characteristic;
receiving a second input in the control system representative of a post-treatment sugar product target specification;
using the control system to determine at least one operating parameter for addition of an additive to the pre-treatment sugar and operating the addition of the additive in accordance with the at least one determined operating parameter, wherein the at least one determined operating parameter is determined from at least:
   the first input,
   the second input, and
   a correlation relating at least the first input and the second input to the at least one operating parameter; and
treating the pre-treatment sugar composition by addition of the additive to produce a post-treatment sugar product with a characteristic that is at or nearer to the target specification than the characteristic of the pre-treatment sugar composition. Optionally, the first input is representative of a pre-treatment sugar composition characteristic. Alternatively, the first input is representative of an additive composition characteristic.

As described elsewhere in the specification, this process can be adjusted so that there is a first input, a second input and a third input used to determine the at least one operating parameter for the addition of the additive.

In an embodiment, after the step of adding the additive to the pre-treatment sugar composition, the process further includes obtaining corresponding output characterisation data from the post-treatment sugar product, and updating the database with the first input and/or third input, the corresponding output characterisation data, and the operating parameter(s) used in processing. In a further embodiment, after the step of subjecting the pre-wash sugar composition to the centrifuge treatment process, the process further includes obtaining corresponding output characterisation data from the pre-treatment sugar product, and updating the database with the alpha input, the corresponding output characterisation data, and the operating parameter(s) used in processing. In a further embodiment, output characterisation data is received and used to update the database after both the centrifuge treatment process and the addition of the additive.

In preferred forms the methods and systems described herein can be used in the production of a sugar product as described in the International Patent application no PCT/AU2017/050782 with the title “Sugar composition” filed by the same applicant or the sugar product as described in patent application no SG 10201807121Q with the title “Sugar composition” filed by the same applicant. The entire disclosure of this document is incorporated herein by reference.

There are multiple further embodiments of the invention including, in an embodiment of the first aspect of the invention, a method for producing a sugar product including:
receiving a first input in a control system representative of a pre-treatment sugar composition characteristic or an additive composition characteristic;
receiving a second input in the control system representative of a post-treatment sugar product target specification;
using the control system to determine at least one operating parameter for addition of an additive to the pre-treatment sugar and operating the addition of the additive in accordance with the at least one determined operating parameter, wherein the at least one determined operating parameter is determined from at least:
   the first input,
   the second input, and
   a correlation relating at least the first input and the second input to
   the at least one operating parameter; and
treating the pre-treatment sugar composition by addition of the additive to produce a post-treatment sugar product with a characteristic that is at or nearer to the target specification than the characteristic of the pre-treatment sugar composition, wherein the addition of the additive occurs at a primary sugar mill. Alternatively, addition of the additive occurs at a sugar refinery.

In embodiments of all aspects of the invention the inputs and specification optionally represent polyphenol content, for example, in an embodiment of the first aspect of the invention, there is provided a method for producing a sugar product including:
receiving a first input in a control system representative of the polyphenol content of a pre-treatment sugar composition or the polyphenol content of an additive composition;
receiving a second input in the control system representative of a post-treatment sugar product target polyphenol content;
using the control system to determine at least one operating parameter for addition of an additive to the pre-treatment sugar and operating the addition of the additive in accordance with the at least one determined operating parameter, wherein the at least one determined operating parameter is determined from at least:
   the first input,
   the second input, and
   a correlation relating at least the first input and the second input to the at least one operating parameter; and
treating the pre-treatment sugar composition by addition of the additive to produce a post-treatment sugar product with a polyphenol content that is at or nearer to the target specification than the polyphenol content of the pre-treatment sugar composition.

In a further example, an embodiment of the third aspect of the invention, there is provided a system for producing a sugar product including:
at least one spray system for adding an additive to a pre-treatment sugar composition to produce a post-treatment sugar product;
at least one sensor for determining one or more of a pre-treatment sugar composition characteristic, an additive characteristic and a post-treatment sugar product characteristic representative of polyphenol content;
a control system configured to determine at least one operating parameter for the at least one spray system based on two or more of:
   a pre-treatment sugar composition characteristic representative of polyphenol content,
   an additive composition characteristic representative of polyphenol content, and
   a target polyphenol content specification of the post-treatment sugar product; and
   a correlation relating the at least one operating parameter to two or more of the pre-treatment sugar composition characteristic, the additive composition characteristic and the target specification;
wherein the control system is further configured to operate the at least one spray system in accordance with the operating parameter.

In the embodiments where the inputs and specification represent polyphenol content:
(i) the polyphenol content can be measured by conducting spectral analysis to measure total polyphenol or tricin content, eg via NIR, or by measuring colour, eg by ICUMSA Units, and/or conductivity; and/or
(ii) the target specification is correlated with a post-treatment sugar having about 20 mg CE polyphenols/100 g carbohydrates to about 45 mg CE polyphenols/100 g carbohydrates, about 46 mg CE polyphenols/100 g carbohydrates to about 100 mg CE polyphenols/100 g carbohydrates (about 37 mg GAE polyphenols/100 g carbohydrates to about 80 mg GAE polyphenols/100 g carbohydrates), or about 60 mg CE polyphenols/100 g carbohydrates (about 50 mg GAE polyphenols/100 g carbohydrates).

In the embodiments where the inputs and specification represent polyphenol content the additive is optionally 500 to 10,000 mg GAE/100 g carbohydrates, 1,000 to 10,000 mg GAE/100 g carbohydrates or 5,000 to 10,000 mg GAE/100 g carbohydrates and preferably a powder. Alternatively, where less polyphenols are required the additive may be 5 to 500, 10 to 250, 100 to 500 or 5-50 mg GAE polyphenols/100 g carbohydrates.

In embodiments of all aspects of the invention the first input and/or third input are received by the control system from a sensor.

In further embodiments of the third aspect of the invention, there is provided a system for producing a sugar product including:
at least one spray system for adding an additive to a pre-treatment sugar composition to produce a post-treatment sugar product;
at least one sensor for determining one or more of a pre-treatment sugar composition characteristic, an additive characteristic and a post-treatment sugar product characteristic representative of polyphenol content;
a control system configured to determine at least one operating parameter for the at least one spray system based on two or more of:
   a pre-treatment sugar composition characteristic representative of polyphenol content,
   an additive composition characteristic representative of polyphenol content, and
   a target polyphenol content specification of the post-treatment sugar product; and
   a correlation relating the at least one operating parameter to two or more of the pre-treatment sugar composition characteristic, the additive composition characteristic and the target specification;
wherein the control system is further configured to operate the at least one spray system in accordance with the operating parameter,

wherein
(i) the system for producing the sugar includes a location for addition of the additive having a pre-treatment sugar composition feed line and the pre-treatment sugar composition feed line has at least one sensor for sensing the pre-treatment sugar composition characteristic as the pre-treatment sugar is fed to the location for addition of the additive; and/or
(ii) the system for producing the sugar includes a location for addition of the additive having an additive feed line and the additive feed line has at least one sensor for sensing the additive characteristic as the additive is fed to the spray system for spraying into the location for addition of the additive.

Optionally, the pre-treatment sugar and additive use the same feed line. Optionally, there is an outlet line for outlet of the post-treatment sugar and the outlet line has a sensor for sensing the post-treatment sugar output characteristic.

In a ninth aspect of the invention there is provided a sugar production plant that includes the above mentioned methods and systems for producing sugar.

In a tenth aspect of the invention, there is provided a sugar product when prepared by a method of the invention as described elsewhere in the specification.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1 is a process flow diagram showing a primary mill sugar process of the prior art.

FIG. 2 is a process flow diagram showing a sugar refining process of the prior art.

FIG. 3 is a process flow diagram showing a smaller purpose built sugar refining plant according to the present invention.

FIGS. 4A to 7 are schematic block diagrams representing a portion of a sugar processing system that can implement an embodiment of the present invention.

FIG. 8 is a total phenolics calibration score plot indicative of the distribution of the sample population in two dimensions.

FIG. 9 is a total phenolics calibration regression plot.

FIG. 10 is a total phenolics calibration plot of explained variance.

FIG. 11 is a total phenolics calibration predicted vs reference plot showing the relationship between the reference data value and the NIR-predicted value.

FIG. 12 is a ICUMSA sugar color calibration score plot indicative of the distribution of the sample population in two dimensions.

FIG. 13 is a ICUMSA sugar color calibration regression plot.

FIG. 14 is a ICUMSA sugar color calibration plot of explained variance.

FIG. 15 is a ICUMSA sugar color calibration predicted vs reference plot showing the relationship between the reference data value and the NIR-predicted value.

FIG. 16 is a tricin calibration score plot indicative of the distribution of the sample population in two dimensions.

FIG. 17 is a tricin calibration regression plot.

FIG. 18 is a tricin calibration plot of explained variance.

FIG. 19 is a tricin calibration predicted vs reference plot showing the relationship between the reference data value and the NIR-predicted value.

FIG. 20 is a graph showing first wash time vs. ICUMSA.

FIG. 21 is a graph showing second wash time vs. ICUMSA.

### DETAILED DESCRIPTION OF THE EMBODIMENTS

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

Further aspects of the present invention and further embodiments of the aspects described in the preceding paragraphs will become apparent from the following description, given by way of example.

All of the patents and publications referred to herein are incorporated by reference in their entirety.

For purposes of interpreting this specification, terms used in the singular will also include the plural and vice versa.

One skilled in the art will recognize many methods and materials similar or equivalent to those described herein, which could be used in the practice of the present invention. The present invention is in no way limited to the methods and materials described.

The inventors of the present invention have developed a process for preparing low GI and/or low GL sugar. The process has advantages in that is it a cost-effective process for preparing healthier sugar that can be applied in a refinery or in a primary mill with massecuite of low polyphenol content.

The term “additive” refers to an additive that changes the composition of the final sugar, for example, by leaving a residue on the sugar or adding an ingredient to the sugar. In some embodiments, the additive includes polyphenols. Pure water is not an additive according to the invention. Neither is acid or base an additive. The additives of the invention, if added to the sugar in the form of a liquid, remain as a remnant ingredient on or in the sugar following drying as opposed to water, acid/base or other solvent that would evaporate during drying.

The term “control system” refers to a manual or partially or fully automated system that receives inputs, considers the inputs in combination with the historical input, operating parameters and/or output information and/or an algorithm developed from historical input, operating parameters and/or output information to determine an appropriate operating strategy.

The term “high glycaemic” refers to a food with a glucose based GI of 70 or more.

The term “low glycaemic” refers to a food with a glucose based GI of 55 or less.

The term “massecuite” refers to a dense suspension of sugar crystals in the mother liquor of sugar syrup. This is the suspension that remains after concentration of the sugar juice into a syrup by evaporation, crystallisation of the sugar and removal of molasses. The massecuite is the product that is washed in a centrifuge to prepare bulk sugar crystals. Massecuite is produced during the production of sugar from both sugar cane and sugar beet. Massecuite from either source is suitable for use in the present invention.

The term “medium glycaemic” refers to a food with a glucose based GI of 56 to 69.

The term “phytochemical” refers generally to biologically active compounds that occur naturally in plants.

The term “polyphenol” refers to chemical compounds that have more than one phenol group. There are many naturally occurring polyphenols and many are phytochemicals. Flavonoids are a class of polyphenols. Polyphenols including flavonoids naturally occur in sugar cane. In the context of the present invention the polyphenols that naturally occur in sugar cane are most relevant. Polyphenols in food are micronutrients that are of interest because of the role they are currently thought to have in prevention of degenerative diseases such as cancer, cardiovascular disease or diabetes.

The term “reducing sugar” refers to any sugar that is capable of acting as a reducing agent. Generally, reducing sugars have a free aldehyde or free ketone group. Glucose, galactose, fructose, lactose and maltose are reducing sugars. Sucrose is not a reducing sugar.

The term “refined white sugar” refers to fully processed food grade white sugar that is essentially sucrose with minimal reducing sugar content and minimal phytochemicals such as polyphenols or flavonoids.

The term “sensor” refers to any means for detecting a characteristic of the pre-treatment sugar, additive or post-treatment sugar. Sensors optionally sense colour, NIR spectra, UV-vis, conductivity or other characteristics.

The term “sugar” refers to a solid that contains one or more low molecular weight sugars such as sucrose. In preferred embodiments, the sugar is a sucrose sugar (ie about 80%, 90% or 95% of the sugar is sucrose).

The term “very low glycaemic” refers to a food with a glucose-based GI of less than half the upper limit of low GI (ie the GI is in the bottom half of the low GI range).

Polyphenol Content Measurement

Polyphenol content can be measured in terms of its catechin equivalents or in terms of its gallic acid equivalents (GAE). Amounts in mg CE/100 g can be converted to mg GAE/100 g by multiplying by 0.81.

Glycaemic Response (GR)

GR refers to the changes in blood glucose after consuming a carbohydrate-containing food. Both the GI of a food and the GL of an amount of a food are indicative of the glycaemic response expected when food is consumed.

GI

The glycaemic index is a system for classifying carbohydrate-containing foods according to how fast they raise blood-glucose levels inside the body. Each carbohydrate containing food has a GI. The amount of food consumed is not relevant to the GI. A higher GI means a food increases blood-glucose levels faster. The GI scale is from 1 to 100. The most commonly used version of the scale is based on glucose. 100 on the glucose GI scale is the increase in blood-glucose levels caused by consuming 50 grams of glucose. High GI products have a GI of 70 or more. Medium GI products have a GI of 55 to 69. Low GI products have a GI of 54 or less. Low GI products are foods that cause slow rises in blood-sugar.

Those skilled in the art understand how to conduct GI testing, for example, using internationally recognised GI methodology (see the Joint FAO/WHO Report), which has been validated by results obtained from small experimental studies and large multi-centre research trials (see Wolever et al 2003).

GL

Glycaemic load is an estimate of how much an amount of a food will raise a person's blood glucose level after consumption. Whereas glycaemic index is defined for each type of food, glycaemic load is calculated for an amount of a food. Glycaemic load estimates the impact of carbohydrate consumption by accounting for the glycaemic index (estimate of speed of effect on blood glucose) and the amount of carbohydrate that is consumed. High GI foods can be low GL. For instance, watermelon has a high GI, but a typical serving of watermelon does not contain much carbohydrate, so the glycaemic load of eating it is low.

One unit of glycaemic load approximates the effect of consuming one gram of glucose. The GL is calculated by multiplying the grams of available carbohydrate in the food by the food's GI and then dividing by 100. For one serving of a food, a GL greater than 20 is high, a GL of 11-19 is medium, and a GL of 10 or less is low.

ICUMSA

ICUMSA is a sugar colour grading system. Lower ICUMSA values represent less colour. ICUMSA is measured at 420 nm by a spectrophotometric instrument such as a Metrohm NIRS XDS spectrometer with a ProFoss analysis system. Currently, sugars considered suitable for human consumption, including refined granulated sugar, crystal sugar, and consumable raw sugar (ie brown sugar), have ICUMSA scores of 45-5,000.

Sugar Processing

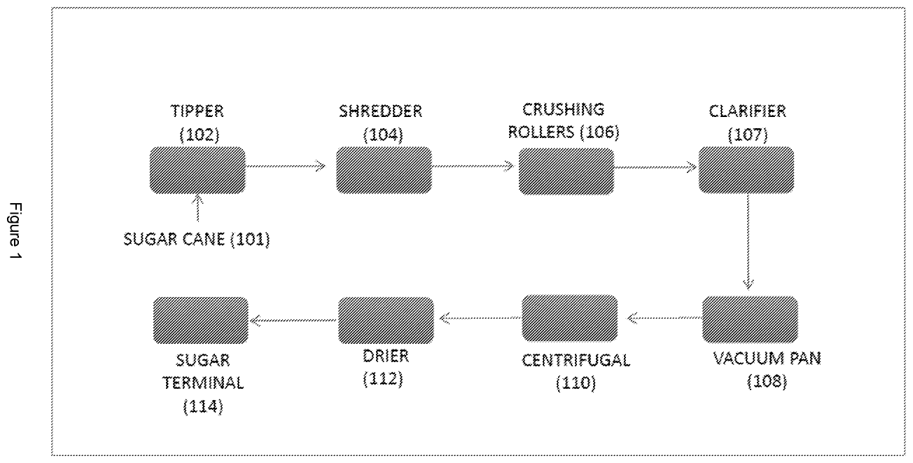

FIG. 1 is a process flow diagram showing a standard primary mill sugar process 100. Briefly, in this process 100, sugar cane 101 passes from a tipper 102 into a shredder 104, before passing through crushing rollers 106. The purpose of this is to extract the sugar containing juice from the sugar cane 101. The sugar containing juice is then further processed such as in a clarifier 107 to removed suspended solids from the juice. The clarified juice is then passed to a vacuum pan 108, where water is evaporated to concentrate the juice into thick syrup including sugar crystals. Sugar crystals are separated from mother liquor using a centrifuge 110 (sometimes colloquially called a “centrifugal” or “fugal” in the field) in a centrifugal washing process. The sugar is then dried in a dryer 112 and stored in a bulk sugar terminal 114 in the form of dark coloured, non-food grade sugar that is about 96-99 wt % sucrose. Further processing of this non-food grade sugar is required to convert the sugar to refined white sugar, which is 99.9 wt % sucrose.

This further processing to form the refined white sugar requires expensive processing steps that typically include: remelting, carbonatation, decolourisation, and filtering. These steps are required to remove the colour components to form a high quality refined white sugar product. Presently, this additional processing typically adds about 33% of the final finished cost.

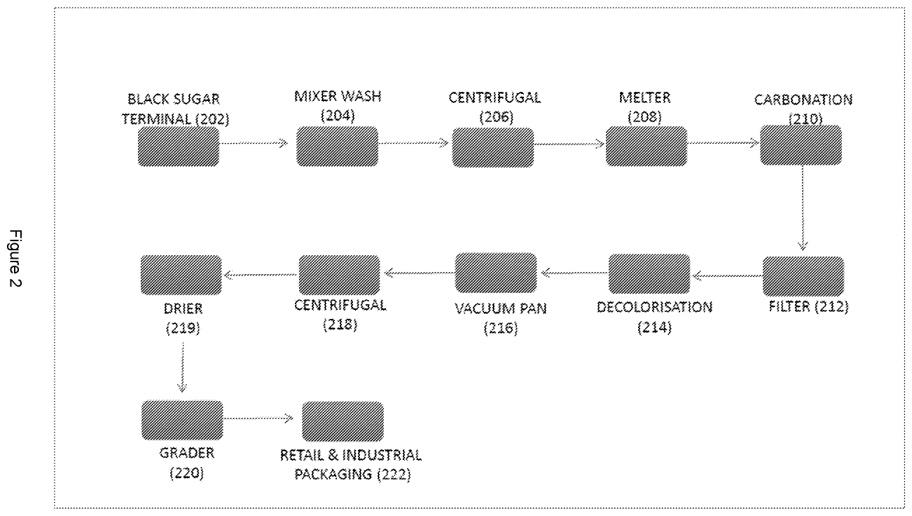

FIG. 2 is a process flow diagram showing an existing sugar refining process 200. Bulk sugar crystals are transported from a bulk sugar terminal 202 to a mixer/washer 204 where the sugar is mixed with heavy syrup. The purpose of this is to dissolve the outer layers of the sugar crystals which typically include a greater level of impurities than within the crystal. This mixture is then fed into a centrifuge 206, for a centrifugal washing process, to further remove impurities from the washed sugar crystals. In some processes, the sugar is then treated using a melter 208 prior to being fed into a carbonation unit 210 where it undergoes a carbonatation process whereby limewater is introduced into a sugar syrup composition which aids in the precipitation of impurities and their subsequent removal using filtration 212. Once the solids have been removed, the syrup may then be decolourised 214 through filtration through a bed of activated carbon or with ion-exchange resin. The sugar is then dried in a vacuum pan 216 and may be further washed in another centrifuge 218 if required. The product is dried 219 before being graded 220 and packaged in industrial bags 222 for shipping.

The inventor has developed a new process that may enable the manufacture of a consistent sugar product. This sugar product can be tailored for industrial, wholesale, foodservice, and retail use. One of the target sugar products is a raw sugar having a low GI rating. However, it will be appreciated that a range of different sugar products of varying specification may be produced. This sugar production process is typically a lower cost process than traditional processes, and generally results in improved product quality (e.g. more consistent specifications) and can also result in lower energy (which also has the benefit of reducing carbon emissions) and water usage.

In a typical batch centrifugal washing process, the process includes at least the steps of loading the basket of the centrifuge with a pre-treatment sugar composition; spinning the centrifuge and spray washing the sugar crystals, and unloading the washed post-treatment sugar product from the centrifuge. These general steps are well known to the skilled person. In some embodiments of the invention, the centrifugal washing process is also controlled and optimised in addition to control and optimisation of the addition of the additive. These tailored properties include, amongst other things: tailored glycaemic index (GI) profile, for example low GI sugars, which can be prepared via a tailored polyphenol content; tailored flavour profiles, which allows speciality sugars to be produced for a specific purpose (such as an ingredient in a food or beverage item), or tailored physicochemical properties. Furthermore, the method allows fewer processing steps, and therefore reduces both capital and operating expenses.

During the centrifugal washing step, the centrifuge is ramped up to steady state at a constant rotational speed. The resultant g-force causes the sugar crystals to form a layer over the vertical walls of the centrifuge basket. Wash water is introduced, such as in the form of spray water, which contacts the exposed surface of the sugar crystals and dissolves the outer layer of the sugar crystals which has a higher level of impurities than the within the sugar crystals. The g-force developed within the centrifuge causes the wash water to permeate through the sugar crystal layer and dissolve further surface impurities from the sugar crystals within the layer. At the end of the washing step, the rotational speed of the centrifuge is ramped down from steady state till rotation ceases. The resultant post-treatment sugar product can then be removed.

There are a number of parameters that can be controlled during operation of the centrifuge, and each of these can impact the properties and composition of the post-treatment sugar product. These parameters include: volume of water used for centrifugal washing; duration of centrifugal washing; temperature of wash water; control of water delivery mechanism, duration, and rate; steady state rotational speed of the centrifuge or g-force; rate at which the rotation speed of the centrifuge is ramped up or down; duration of ramping the speed of the centrifuge up, down, and operating at steady state.

Given the above, the inventor has found that by assessing the quality of the pre-treatment sugar composition it is possible to determine a strategy for addition of an additive, for example, in the form of setting operating parameters for spraying the additive onto the pre-treatment sugar, to provide a post-treatment sugar product having desired characteristics. This feed forward control system enables tighter control of the addition of the additive than traditional sugar manufacture (which does not use a control system) or the more recent feedback control systems, which significantly reduces variability of the raw product, so a consistent specification can be achieved. The system can be further improved by assessing the quality of the post-treatment sugar and using the information available on the pre-treatment sugar, additive, operating parameters for addition of the additive and/or the post-treatment sugar characteristics to refine the correlation used to set the operating parameters. Where the system includes the feedforward control features of the invention and these feedback features, the control system is a closed loop control system. The feed forward and/or closed loop systems can be automated as discussed elsewhere and real-time adjustment of the machinery for addition of the additive and/or washing of the pre-wash sugar can be achieved using sensors to further optimise the efficiency and reproducibility of achieving a post-treatment sugar that meets the target specification despite variations in the quality of the pre-wash sugar, the pre-treatment sugar and the additive.

As above, these characteristics may be in the form of a specific GI, colour, or flavour profile. By way of example, a specialty sugar having a particular GI profile, colour, and flavour profile may be desired. To produce this product, an analytical process such as NIR may be used to derive a spectrum that is indicative of phenol or flavonoid types and concentrations in the pre-treatment sugar composition. In another example, phytochemicals are directly (or indirectly) standardized from a pre-treatment sugar composition (such as massecuite) to the post-treatment sugar product. In each case, appropriate process operating parameters for the centrifuge can then be adopted to produce the specialty product. These process operating parameter(s) can be determined by assessing the input characteristics and the desired output characteristics in conjunction with a database that includes historical production data (e.g. input characteristics with corresponding output characteristics and the centrifuge operating parameter(s)). Thus, the system is in effect a feed forward control system which assesses an input quality and determines process operating parameter(s) for the addition of the additive are based, in part, on historical empirically derived data. By further including some form of analysis downstream, such as a further NIR spectrometer, the quality of the post-treatment product can be evaluated. The database may then be updated with the input, output, and centrifuge process operating parameter(s) from this iteration. A similar approach can be used to control the centrifugal washing process during the preparation of the pre-treatment sugar.

The correlation of one or more input characteristics and/or output characteristics and one or more processing parameters in the database is particularly advantageous during the processing of the early batches of a bulk load of pre-treatment sugar product and/or a bulk load of additive. As will be appreciated each bulk load of additive can be different to the last. There can also be variation in the pre-treatment sugar product, although the variation will be less where the pre-treatment sugar is prepared by a controlled centrifugal wash. In prior art systems the parameters used for the first batch of a new bulk load were based purely on operator skill or some standard operating procedure. However, in embodiments of the present invention the measured input characteristic(s) of the first batch can be used to choose more reliable operating parameters, which can be refined over time with subsequent batches.

Given the above, one element of this technology is the use of a new closed loop NIR sugar analysis system that incorporates a control algorithm. In a preferred embodiment the method uses a heuristic algorithm to produce a sugar product of desired composition. The algorithm is able to determine and implement an operating strategy for spray treatment of a pre-treatment sugar composition based on the composition of the pre-treatment sugar and/or the additive and the desired or target composition of the sugar product after addition of the additive. The operating strategy will include at least one operating parameter for the addition of the additive and is determined from a database that includes historical information regarding input compositions, corresponding output compositions, and the corresponding process conditions. By continuing to measure and record respective inputs, outputs, and process conditions, the database that the algorithm draws upon is expanded with additional data which further increases the reliability of the process control system.

As discussed above, one advantage of this increased level of process control is that fewer processing steps are required to produce a sugar product. FIG. 3 is a process flow diagram of a purpose-built plant for processing raw sugar that includes an optional controlled centrifugal washing system and a controlled spray system. As can be seen, in this process, bulk sugar crystals are transported from a bulk sugar terminal 302 to a mixer/washer 304 where the sugar is mixed with heavy syrup; this is then fed into a centrifuge 306 for washing of the sugar crystals. Once washing in the centrifuge 306 is completed, the sugar crystals are separated from the liquor. Optionally, the sugar crystals are dried before addition of the additive. The additive is then added to the sugar crystals 307 and the sugar crystals then fed to a dryer 308, where the crystals are dried. The additive may be added in the centrifuge following the wash. The additive may be added separate from the centrifuge, as depicted in FIG. 3. The sugar crystals are then graded 310 and packaged 312 for shipping.

Sensors may be included at various stages throughout the process to affect the desired control.

In one example, the system may include at least two sensors, a first sensor located upstream of the addition of the additive and a second sensor located downstream of the addition of the additive. The first sensor is preferably located adjacent to the inlet via which the pre-treatment sugar is added to the container for addition of the additive or adjacent to the inlet via which the additive is added (depending on whether the first input is a characteristic of the pre-treatment sugar or the additive) so that it can determine a characteristic of the pre-treatment sugar composition or additive before or as it enters the container for addition of the additive). Where the additive is added in the centrifuge after the wash, the first sensor is optionally located to sense the pre-treatment sugar in the centrifuge after removal of the wash liquid or at the inlet for the additive to the centrifuge. The second sensor is preferably located adjacent to the outlet of the container for addition of the additive so that it can determine a characteristic of the post-treatment sugar product as it leaves the centrifuge.

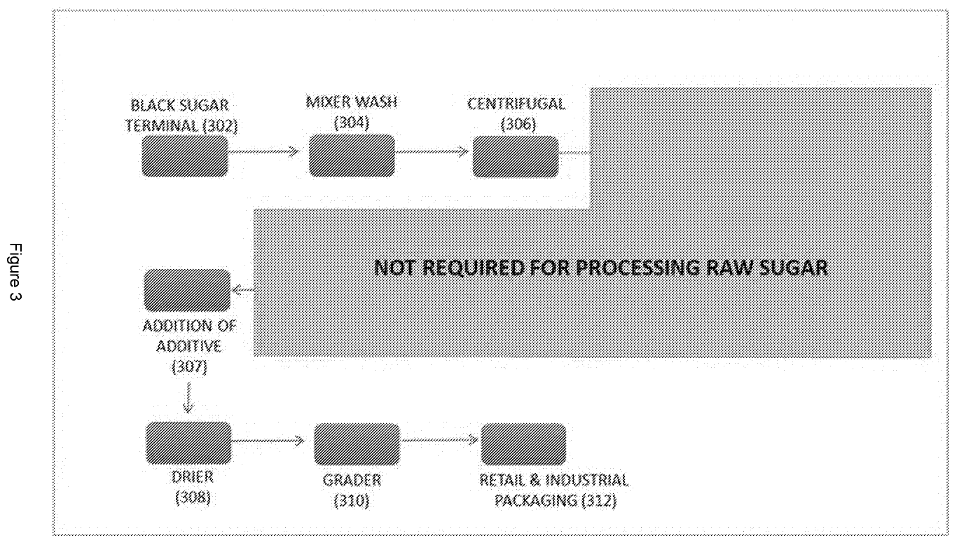

FIG. 4 illustrates embodiments of this example. In FIG. 4A, the system 400 includes a location for addition of an additive 402 having a pre-treatment sugar composition feed line 404, an additive feed line 405 and an outlet line 406 for off-take of the post-treatment sugar product. In some embodiments, the additive may use the same feed line as used for the pre-treatment sugar. The feed line 404 includes a sensor 408 for measuring a characteristic of the pre-treatment sugar composition. As discussed previously, a range of different sensors may be used. However, in this example, the sensor 408 is an NIR spectrometer for detecting the presence of tricin. Data from the sensor 408 is fed to a control system 410, and the control system 410 determines an appropriate operating parameter with which to operate the addition of the additive 402 in order to obtain a post-treatment sugar product with desired characteristics or a desired profile. This operating parameter may be empirically determined from a database of stored historical inputs, outputs, and operating parameters; or the operating parameter may be based on an equation that determines an operating parameter from input characterisation data. Such an equation may, for example, be empirically derived from historical data. In any event, the pre-treatment sugar composition is then fed into the container for addition of an additive 402, where the additive is added according to the operating parameter, for example, where the additive is added by a spray system, spray time, spray pressure etc, as determined by the control system 410 to obtain the desired characteristics or profile. Once addition of the additive is completed, the post-treatment sugar product passes out of the container for addition of the additive 402. A sensor 412 on the outlet line 406 measures an actual characteristic or profile of the post-treatment sugar product, and relays this information back to the control system 410. The control system 410 can compare the actual characteristics or profile of the post-treatment sugar product against the desired characteristics or profile and optionally perform a number of tasks to improve process control. The control system 410 may update the database with the input, desired output, actual output, and operating parameter used to provide the system with additional historical data upon which to determine a future operating parameter. Alternatively, or additionally, the control system 410 may alter the form of the equation used to determine the operating parameter; for example, if the sensor 412 on the outlet line 406 determines that the concentration of tricin is too high, then the control system 410 may adjust the equation such that future additions of additive are shortened (and/or adjust other operating parameters in an appropriate manner). Alternatively, or additionally, the control system 410 may act to apply a change to the operating parameter in order to improve the output. By way of example, if the sensor 412 on the outlet line 406 determines that the concentration of tricin is too high, then the control system may simply decrease the spray time (or alter another operating parameter in the appropriate manner). e.g. by adding a fixed value to the spray time, (e.g. 0.1 second), by multiplying the previously determined spray time by a predetermined fashion, or other numerical adjustment method.

In the system 400 of FIG. 4A, there are no unit processes between the sensor 408 on the inlet line 404 and the location for addition of an additive 402, and similarly no unit processes between the location for addition of an additive 402 and the sensor 412 on the outlet line 406. However, it will be appreciated that in certain embodiments, there may be one or more unit processes performed between sensors 408 or 412 and the centrifuge 402. By way of example, the post-treatment sugar product may be subjected to a drying process after treatment in the centrifuge 402, but prior to passing sensor 412.

In this embodiment, a sensor may not be needed on the additive feed line because the additive has a known specification such that sensing the characteristic of the pre-treatment sugar is sufficient to determine a suitable operating parameter for addition of the additive.

FIG. 4B provides a similar system except there is no sensor 408. Instead there is a sensor 409 on the additive feed line 405 and no unit processes between the sensor 409 and the location for addition of an additive 402. Data from the sensor 409 is fed to a control system 410.

In this embodiment, a sensor may not be needed on the pre-treatment sugar feed line because the pre-treatment sugar has a known specification such that sensing the characteristic of the additive is sufficient to determine a suitable operating parameter for addition of the additive.

In an alternative embodiment to the embodiment described above in relation to FIGS. 4A and 4B, there are three sensors. A sensor 408 for measuring a characteristic of the pre-treatment sugar composition, a sensor 409 on the additive feed line and a sensor 412 on the outlet line.

In an alternative embodiment to the embodiment described above in relation to FIGS. 4A and 4B, there are two sensors. A single sensor 408/409 is used for measuring a characteristic of the pre-treatment sugar composition and a characteristic of the additive because both the pre-treatment sugar and the additive are added via the same feed line (at different times) and a sensor 412 on the outlet line. Alternatively, a single sensor 408/412 is used for measuring a characteristic of the pre-treatment sugar composition and a characteristic of the post-treatment sugar composition because the post-treatment sugar exist the container via the pre-treatment sugar inlet and sensor 409 for measuring a characteristic of the additive. A similar embodiment can occur where the post-treatment sugar exist via the additive inlet. It is possible for there to be a single sensor 408/409/412 to sense one or more of the pre-treatment sugar characteristic, the additive characteristic and/or the post-treatment sugar characteristic because the pre-treatment sugar and additive are added via the same inlet and the post-treatment sugar exits the container via the same inlet.

As noted above the sensors 408 and 412 or 409 and 412 can measure any suitable pre or post-treatment sugar composition characteristics. Table 1 below sets out several exemplary sensor configurations that may be used in some embodiments.

TABLE 1

| Example | | |
|---|---|---|
| configuration | Sensor 408/409 | Sensor 412 |
| A. | colour | NIR spectra |
| B. | colour | UV-vis |
| C. | colour | colour |
| D. | colour | conductivity |
| E. | NIR spectra | NIR spectra |
| F. | NIR spectra | UV-vis |
| G. | NIR spectra | colour |
| H. | NIR spectra | conductivity |
| I. | UV-vis | NIR spectra |
| J. | UV-vis | UV-vis |
| K. | UV-vis | colour |
| L. | UV-vis | conductivity |
| M. | conductivity | NIR spectra |
| N. | conductivity | UV-vis |
| O. | conductivity | colour |
| P. | conductivity | conductivity |

In an alternative example, the system may include a single sensor arranged so that it is able to determine a characteristic of the pre-treatment sugar composition before treatment by addition of an additive. In another alternative example, the system may include a single sensor arranged so that it is able to determine a characteristic of the additive before treatment addition of the additive to the pre-treatment sugar.

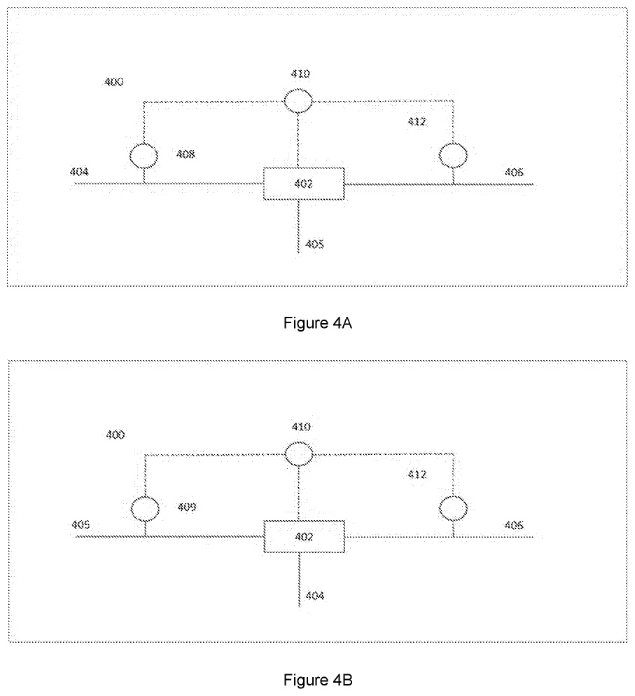

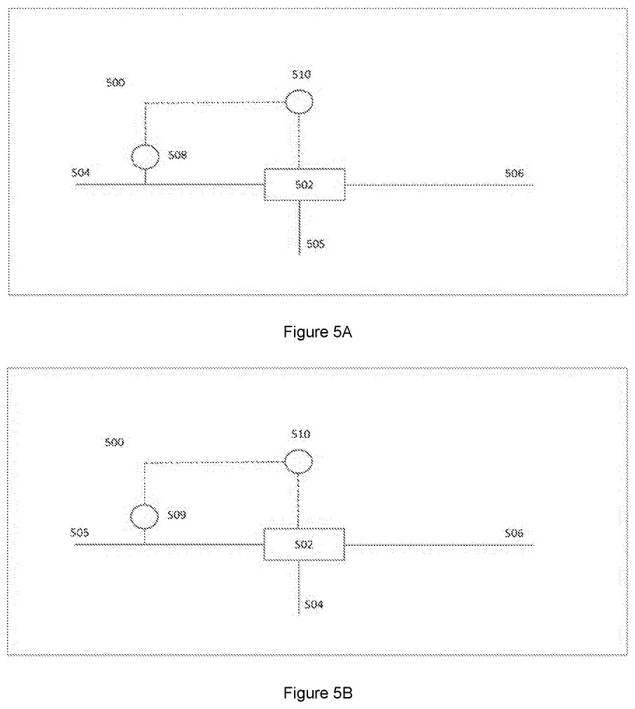

In one such embodiment, illustrated in FIG. 5A, the system 500 includes a location for addition of the additive 502 having a pre-treatment sugar composition feed line 504, an additive feed line 505 and an outlet line 506 for off-take of the post-treatment sugar product. In some embodiments, the additive may use the same feed line as used for the pre-treatment sugar. The feed line 504 includes a sensor 508 for measuring a characteristic of the pre-treatment sugar composition. As with the embodiment of FIG. 4, data from the sensor 508 is fed to a control system 510, and the control system 510 determines an appropriate operating parameter with which to add the additive 502 in order to obtain a post-treatment sugar product with desired characteristics or a desired profile. This system 500 does not include a sensor on the outlet line 506. Thus, this system 500 is provided with no direct means of quality assessment or quality control. This system 500 may be appropriate in a situation where the control system 510 includes a robust repository of historical data to draw upon, and/or a robust equation for determining the operating parameter of the centrifuge 502.

In such a system a further sensor (not shown) can be placed on the outlet line from time to time to test the post-treatment sugar product to thereby test that the correlation being used to determine the operating parameter(s) of the centrifuge is still accurate. Batch testing could also be used for this process

FIG. 5B provides a system similar to that of FIG. 5A except there is no sensor 508. Instead there is a sensor 509 on the additive feed line 505. Data from the sensor 509 is fed to a control system 510.

In an alternative embodiment, there are two sensors. A sensor 508 for measuring a characteristic of the pre-treatment sugar composition and a sensor 509 on the additive feed line.

Alternatively, a single sensor 508/509 is used for measuring a characteristic of the pre-treatment sugar composition and a characteristic of the additive (because both the pre-treatment sugar and the additive are added via the same feed line).

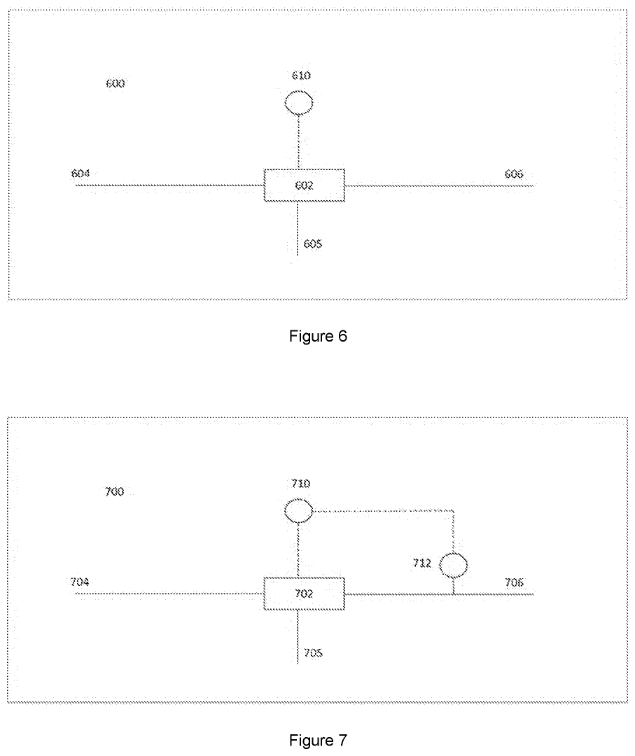

In another such embodiment, illustrated in FIG. 6, the system 600 includes a sensor (not shown) located within the location (eg container) for addition of the additive 602. This sensor may be located underneath the pre-treatment sugar composition on an inlet as the pre-treatment sugar composition flows into the location for addition of the additive 602. Alternatively or in addition a sensor may be located underneath the additive on an inlet of the additive as the additive flows into the location for addition of the additive to the pre-treatment sugar 602. An NIR sensor is suitable for this application. Alternatively the sensor may be placed above the centrifuge 602 to monitor a parameter, such as real time sugar colour, as the sugar composition is being washed. A UV-vis sensor is suitable for this application.

This sensor communicates with the control system 610 to determine an operating parameter for the addition of the additive. The sensor may be used to determine a characteristic of the pre-treatment sugar composition once the sugar composition is in the location for addition of the additive 602 (rather than from feed line 604 or feed line 605) and/or a characteristic of the post-treatment sugar product in the location of the addition of the additive but after processing (rather than from the outlet line 606). Additionally, the sensor may provide characterisation data during processing of the sugar. Thus, this embodiment also offers the advantage that the sensor may be used to provide real-time sensing and reporting during operation of the centrifuge 606. The control system 610 may use the data provided by the sensor to improve process control in a similar manner to that discussed in the embodiment of FIG. 4. In addition to a sensor positioned to determine a characteristic of the pre-treatment sugar product and post-treatment sugar product a further sensor may be included to determine a characteristic of the additive. The further sensor may be in the feed line for the additive or in the inlet to the location for addition of the additive.

In still another embodiment, illustrated in FIG. 7, the system 700 includes a location for addition of an additive 702 having a pre-treatment sugar composition feed line 704, an additive feed line 705 and an outlet line 706 for off-take of the post-treatment sugar product. In some embodiments, the additive may use the same feed line as used for the pre-treatment sugar. In this embodiment, the process does not necessarily include a feedforward sensor for providing the first input representative of the pre-treatment sugar composition characteristic to the control system 710. Instead, the control system 710 receives the first input via an alternative route. By way of example, the pre-treatment sugar characteristic may be measured at an off-site location, such as at a facility where the sugar cane or other unrefined feed materials are harvested and potentially subjected to initial processing to form the feed pre-treatment sugar composition of the method of the present invention. In such cases, the pre-treatment sugar characteristic may be measured off-site and transmitted from this off-site location (such as via the internet or other telecommunications network) to the control system 710, such that when the pre-treatment sugar composition is delivered for treatment according to the present invention, the first input has been received by the control system 710. Alternatively a first input representing a characteristic of the additive may be measures at an off-site location, such as a facility where the additive was prepared and transmitted from that location to the control system 710. In some embodiments, the control system receives both a first input representing a characteristic of the pre-treatment sugar and a third input representing a characteristic of the additive.

In a further embodiment, processes according to the present invention are run in parallel. In this case, a large batch of a pre-treatment sugar composition is subdivided into smaller batches. These smaller batches are then processed in different parallel process trains. This can occur where there is a stock pile of the pre-treatment sugar composition that is significantly larger than the batch size that can be accommodated by a single process line. In this embodiment, a first process train has a sensor for measuring a characteristic of the pre-treatment sugar composition or the additive (such as sensor 408, 409, 508 or 509 in FIGS. 4, 5, or as described in relation to FIG. 6) and other process trains do not include this sensor. Instead, the control systems on these other process trains receive the pre-treatment sugar characteristic and/or additive characteristic from the sensor on the first process train.

Irrespective of the mechanism by which control system 710 receives the pre-treatment sugar characteristic and/or additive characteristic, in this embodiment, once processing in the location for addition of the additive 702 is completed, the post-treatment sugar product passes out of that location 702. A sensor 712 on the outlet line 706 measures an actual characteristic or profile of the post-treatment sugar product, and relays this information back to the control system 710. The control system 710 can compare the actual characteristics or profile of the post-treatment sugar product against the desired characteristics or profile and optionally perform a number of tasks to improve process control. As discussed in relation to embodiment of FIG. 4, the control system 710 may update the database with the input, desired output, actual output, and addition of the additive parameter to provide the system with additional historical data upon which to determine a future operating parameter. Alternatively, or additionally, the control system 710 may alter the form of the equation used to determine the operating parameter.

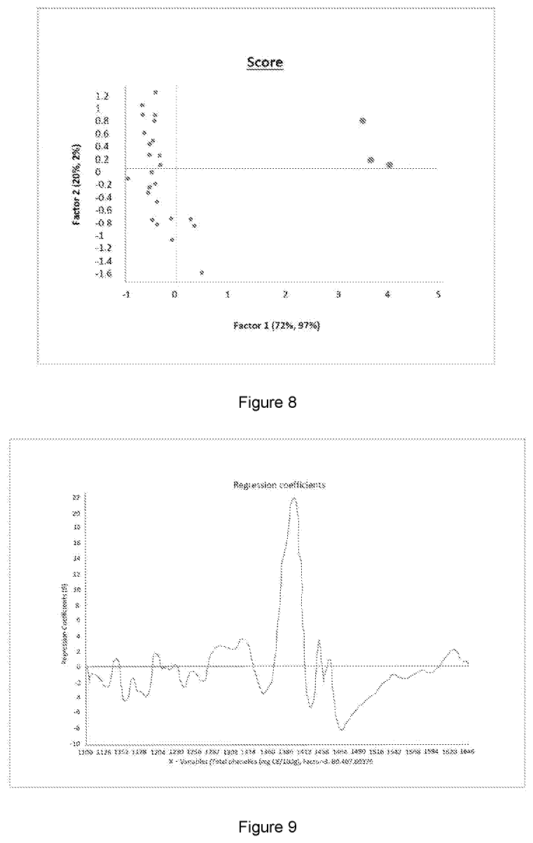

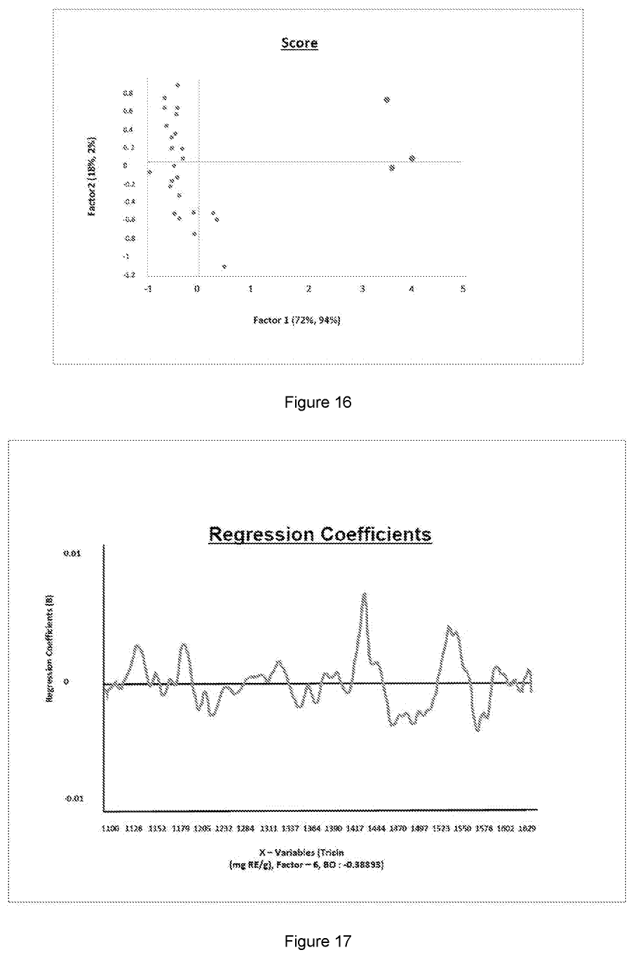

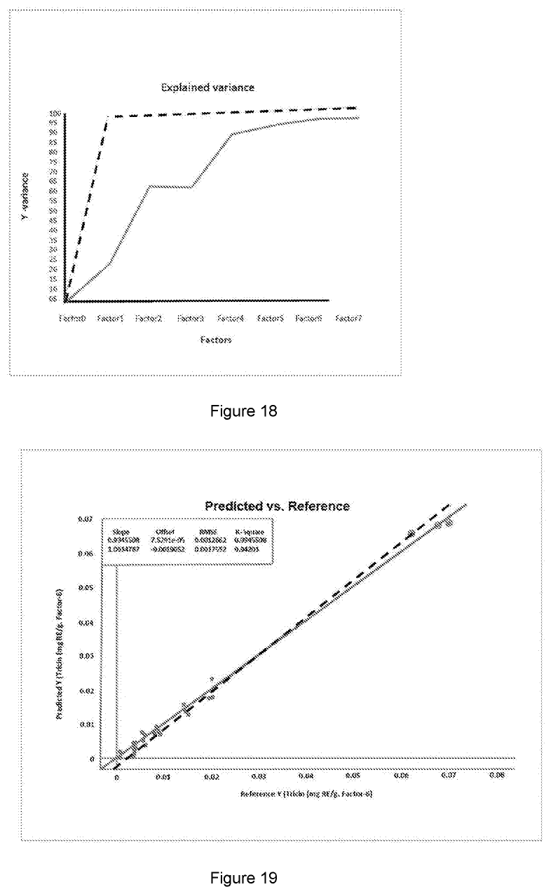

As noted above the target specification could be expressed in terms of any directly measurable characteristic of the pre and/or post treatment sugar products or an alternatively a physico-chemical property which can be correlated with the measured characteristic. FIGS. 8 to 19 show example data obtained using NIR analysis of sugar samples to illustrate that NIR measurement can be used to perform characterisation of a sugar product (either or both at pre- or post-processing by centrifugal washing) and this can be used to target a post-centrifugal washing sugar product target specification (ie the pre-treatment sugar of some embodiments). In these examples, a correlation is demonstrated between NIR measurements and polyphenols, tricin and colour that demonstrates that NIR could be used to directly evaluate product composition in real-time compared to specifications expressed in these parameters. And accordingly that process control could be performed using such NIR measurement techniques.

As will be appreciated by those skilled in the art sugar refineries or mills may be paid differential rates depending on the specification of sugar produced. For example, producing sugar complying with a first specification may attract a different price than sugar produced to a second specification. For example first a specification may be defined by a buyer (e.g. a customer or national sugar board or the like) which sets a target ICUMSA value of less than 1800, for which a first price is paid per tonne, but a second specification may be defined with an ICUMSA value of less than 2500, but attract a lower price. The extent of compliance may also change the price paid for post-treatment sugar products, for example producing sugar in batches with properties more tightly grouped around a specification may attract higher prices or bonus payments, e.g. the first specification may pay a bonus for, e.g. every batch of sugar which has an ICUMSA between 1700 and 1800, or for every day of production where the average ICUMSA over all batches lies between 1700 and 1800. The inventor has previously observed that even with such payment processes in place, the ICUMSA values from samples taken over 20 successive days of a production at the same mill can vary by almost 50%. Such production methods thus can be seen as producing sugar with low batch to batch consistency and a wide statistical distribution of sugar characteristics.

Certain embodiments of the present invention seek to provide either a system or a method that is able to be used in a sugar production process to improve batch-to-batch consistency in production, which may assist refiners and millers of sugar to achieve such specifications with eg 10% batch to batch variation allowance. Such an improvement could in some instances result in a tightening of the statistical distribution of post processed sugar characteristics around a desired target specification. This may allow the refinery or mill to more consistently sell their product for an optimal price, and/or minimise production for a certain post processed sugar product (e.g. by avoiding unnecessary washing etc.) within the specification. Moreover, with some instances of the methods and systems described herein, the possibility to produce specialty sugars defined by a user's target specification could be realised. For example, a food manufacturer may require an ingredient which is a sugar product with an average ICUMSA colour within a set band—say 1900-2000, and a certain proportion of the sugar within a broader band—say 60% of the sugar with an ICUMSA of 1750 to 2150. As noted above, specialty sugars could be defined in terms of other measurable physicochemical properties, e.g. Tricin, polyphenols, conductivity or the like, or some characteristic correlated with these measurable properties.

### EXAMPLES

### Example 1—Control of a Centrifugal Wash Cycle to Control Properties of a Sugar

Sample Collection

27 sugar samples were produced by Mill 1 and Mill 2. Approximately 100 g of raw sugar was sampled using screw capped plastic bottles from the finished product conveyor. Reference data were obtained by wet chemistry or traditional methods and a correlation of these results with measured NIR spectra was performed.

Reference Data

Polyphenol Analysis

40 g of raw sugar sample was weighed into a 100 ml volumetric flask. Approximately 40 ml of distilled water was added and the solution was agitated until the sugar was fully dissolved before solution was made up to final volume with distilled water. The polyphenol analysis was based on the Folin-Ciocalteu method.

In brief, a 50 μL aliquot of appropriately diluted raw sugar solution was added to a test tube. 650 μL of ultrapure water was added and mixed. 50 μL of Folin-Ciocalteu reagent was added and mixed. After 5 minutes 500 μL of 7% Na₂CO₃ solution was added with mixing. After 90 minutes at room temperature the absorbance was read at 750 nm.

The standard curve for total phenolics was prepared using catechin standard solution (0-250 mg/L). Sugar analysis results were expressed as milligrams of catechin equivalent (CE) per 100 g raw sugar.

Colour Analysis

Colour was analysed according to the Sugar Research Australia Standard Analytical Method 33 (2001).

In brief, 20 g of raw sugar was accurately weighed into a 100 ml volumetric flask; approximately 50 ml of distilled water was added and agitated until sugar dissolved. 10 ml of 0.2M MOPS (3-(N-morpholino)propanesulfonic acid) buffer solution (pH 7) was added to flask and the solution made to volume with distilled water. A reference solution was made with the addition of 10 ml MOPS buffer to a 100 ml volumetric flask and made to the mark with distilled water. Each sample solution and reference solution was filtered using 0.8 μm prefilter connected to a 0.45 μm membrane filter (Millipore, Millex HA). Absorbance of the filtered sugar solution was measured at 420 nm using the reference solution as the blank. The ICUMSA colour was calculated.

ICUMSA colour=(A420/concentration in g/ml)×1,000

Results

Comparison to NIR Readings

NIR analysis was performed with a ProFOSS Direct Light NIR spectrophotometer. The Instrument read head was mounted on a vibration damping arrangement and installed within a mounting enclosure for continual analysis of the moving sugar process stream.

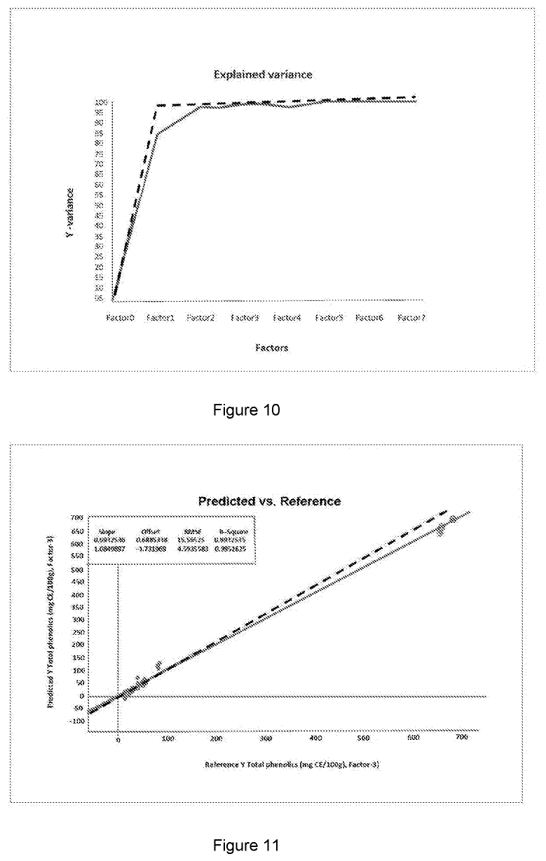

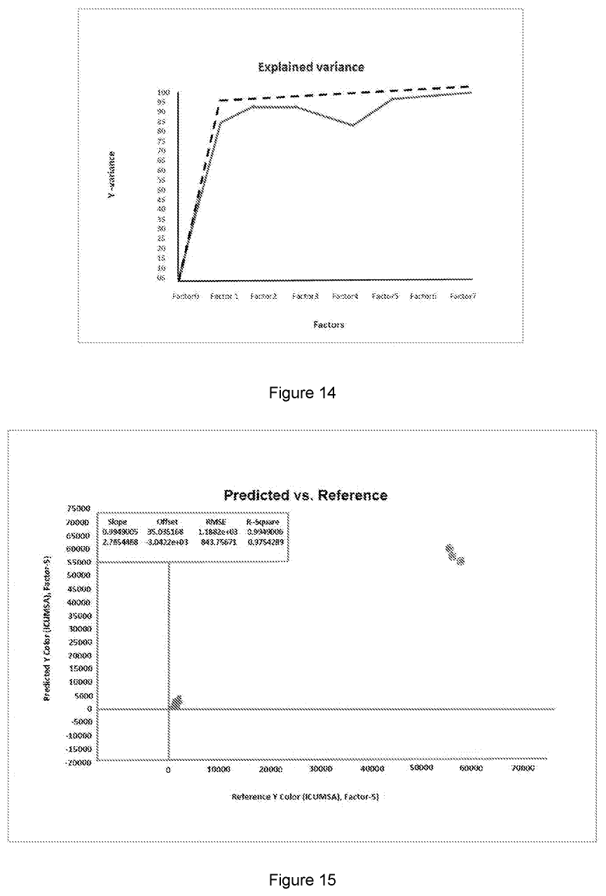

FIGS. 8 to 19 show the calibration parameters of each model and indicate validation performance. Partial least squares (PLS) regression calculates new planes in multivariate space that describe the maximum (residual) variance in the data. These are called factors or principal components. The plot of explained variance (see FIGS. 10, 14 & 18) shows the percentage of the total variation in Y explained by the model with the inclusion of successive factors. The calibration dataset is shown in dashed-line and the validation dataset in solid-line.

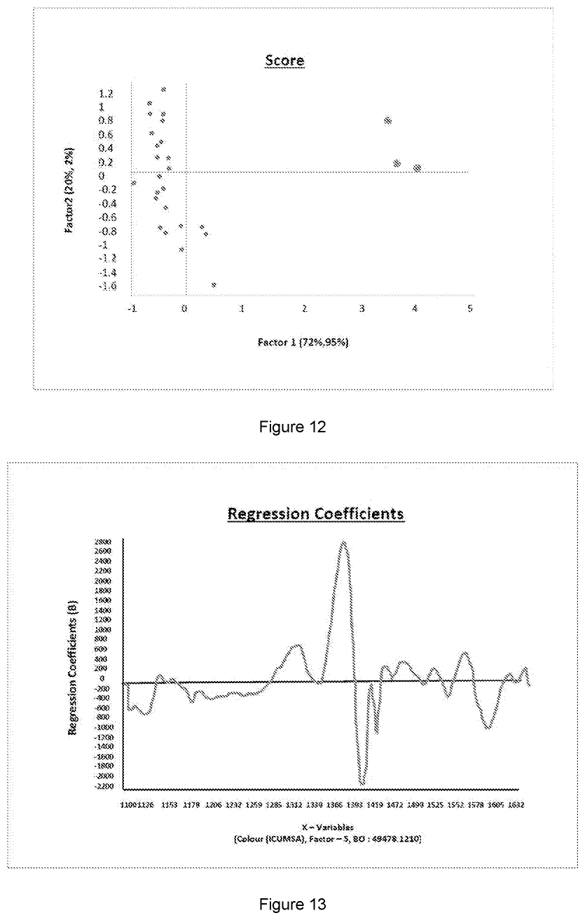

The scores plots (see FIGS. 8, 12 & 17 are indicative of the distribution of the sample population in two dimensions. In this case, Factor 1 and Factor 2 are plotted against each other and represent 97% and 2% of the (residual) variability in the sample set, respectively. Samples that lie close to each other in the scores plot are considered to be similar and those far apart are different from one another.

The regression coefficients (see FIGS. 9, 13 & 18) are also known as the b-vectors or eigenvectors and represent the equation of the model. Multiplying the X-matrix, which is the spectral data for new samples, by the b-vector produces a matrix of predicted Y-values (analyte of interest, e.g. total phenolics). Comparing the regression coefficient with the scores plot assists in identifying which wavelengths (X-variables) are most responsible for the distribution of the samples in the scores plot. Wavelength regions with high regression coefficients represent variable most responsible for the distributions in the scores plot.

The predicted vs reference plots (see FIGS. 11, 15 & 19) show the relationship between the reference data (wet chemistry or traditional methods) value and the NIR-predicted value for a particular analyte. The solid line is the regression of the predicted values against the reference values for the calibration set (top line of results such as slope) and the dashed line represents the same for the validation dataset (bottom line of results such as slope). Table 2 illustrates the correlation achieved with the present set up.

TABLE 2

| | Calibration | | | | Validation | | | |
|---|---|---|---|---|---|---|---|---|
| Model | n | SEC | R² | Factors | n | SEP | Bias | R² |
| Total phenolics | 27 | 4.1 | 0.99 | 5 | 6 | 2.1 | −1.2 | 0.98 |
| Colour | 27 | 92 | 0.98 | 7 | 6 | 162 | −157 | 0.67 |
| Tricin | 27 | 0.0014 | 0.94 | 4 | 6 | 0.0007 | −0.0019 | 0.86 |

This example demonstrates a statistically significant correlation exists between NIR, colour and polyphenols including tricin. This method is therefore useful for a rapid on-line measuring tool for feed forward and feedback purposes in processing sugar.

As will be appreciated, a sugar mill or sugar refinery may include a plurality of centrifuges. In some embodiments of the present invention all centrifuges can be treated in the same way, and the same operating characteristic used for each. This is less accurate, but requires fewer sensors. However, in other embodiments each centrifuge can be provided with a sensor system to measure at least one characteristic of the pre-treatment sugar composition and a corresponding characteristic of a post-treatment sugar composition. This results in increased accuracy. Such sensors may be those previously described, such as colour, NIR, or UV-vis sensors. In further embodiments, either the input or output sensing could be common to more than one centrifuge (e.g. use a common input sensor at a mingler/header tank) but the other ie the output sensing, if the input is a common sensor, (or the input sensing, if the output sensor is a common sensor) can be performed with dedicated sensor(s). In cases where a centrifuge has dedicated output sensing of its post-treatment sugar product, and its own database (or sub-database) of corresponding operating parameters, the present system is capable of accommodating the idiosyncratic behaviour of each centrifuge to achieve more consistent overall output. The use of dedicated input sensing better enables an embodiment to accommodate batch by batch variations in pre-treatment sugar composition.

Near infra-red spectroscopy has been established as a reliable method for analysing processed sugar cane. This example convincingly demonstrates a statistically significant correlation exists between NIR, colour and polyphenols including tricin. This method is therefore useful for a rapid on-line and/or offline measuring tool for feed forward and feedback QA/QC purposes in making low GI sugars.

Control of Output Quality by Adjusting the Centrifuge Wash Cycle

The following example illustrates the effect of controlling wash time on the ICUMSA and total phenolics of a sugar composition. A controlled addition of an additive may be similarly developed. In this example, ten samples of massecuite were washed according to centrifugal wash processes outlined in Table 3 below to produce a raw sugar.

As can be seen, different wash strategies were employed for different massecuite samples. By way of example, for sample M1, the massecuite was exposed to a first wash at 700 RPM for 2 seconds followed by a second wash at 900 RPM for 2 seconds before being subjected to a final spin at 1100 RPM for 5 seconds. Samples M2 to M10 were similarly subjected to various wash strategies as outlined in Table 3. The purpose of the different first and second wash times for these samples is to build up a model based on the raw sugar results.

TABLE 3

| | Centrifuge Settings | | | | |
|---|---|---|---|---|---|
| Sample | 1st wash | 2nd wash | Final spin | Total Wash | Av. Colour |
| ID | sec/rpm | sec/rpm | sec/rpm | time (sec) | (ICUMSA) |
| M1 | 2/700 | 2/900 | 5/1100 | 4 | 1852.405 |
| M2 | 2/700 | 2/900 | 5/1100 | 4 | 1725.04 |
| M3 | 1/700 | 1/900 | 5/1100 | 2 | 1801.105 |
| M4 | 3/700 | 3/900 | 5/1100 | 6 | 1251.92 |
| M5 | 2/700 | 2/900 | 5/1100 | 4 | 1217.08 |
| M6 | 1/700 | 1/900 | 5/1100 | 2 | 1769.715 |
| M7 | 3/700 | 3/900 | 5/1100 | 6 | 1150.795 |
| M8 | 2/700 | 1/900 | 5/1100 | 3 | 1369.815 |
| M9 | 2/700 | 0/900 | 5/1100 | 2 | 1387.335 |
| M10 | 1/700 | 0/900 | 5/1100 | 1 | 1414.785 |

Table 4 lists the initial total phenolics of the massecuite and the final total phenolics of the raw sugar for samples M1 to M10.

TABLE 4

| | Total phenolics raw sugar | Total phenolics massecuite |
|---|---|---|
| | (mg CE polyphenols/100 g | (mg CE polyphenols/100 g |
| Sample ID | carbohydrates) | carbohydrates) |
| M1 | 23.1 | 316.8 |
| M2 | 24.3 | 312.0 |
| M3 | 25.8 | 287.6 |
| M4 | 18.6 | 291.8 |
| M5 | 20.5 | 314.6 |
| M6 | 24.1 | 301.8 |
| M7 | 17.1 | 277.3 |
| M8 | 19.5 | 262.3 |
| M9 | 18.2 | 305.4 |
| M10 | 23.6 | 314.7 |

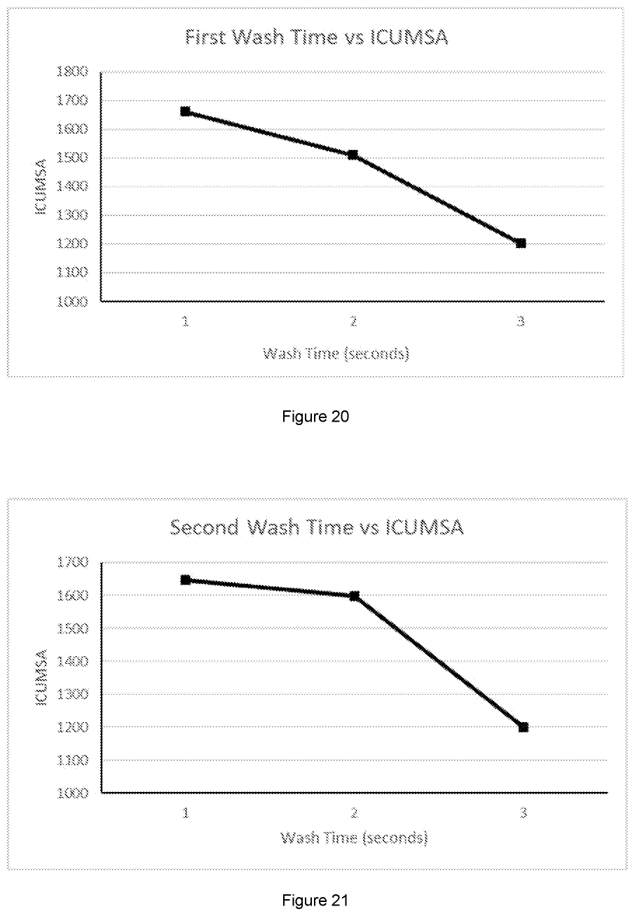

FIG. 20 is a graph first wash time vs. ICUMSA, and FIG. 21 is a graph showing second wash time vs. ICUMSA. As can be seen from FIGS. 20 and 21, there is a correlation between wash time and the ICUMSA of the raw sugar. This can be used to select appropriate first and second wash times for a particular massecuite to produce a raw sugar having a total phenolics concentration in the desired range. The robustness of the correlation, and thus operation of the centrifuge, can be improved by adding further data sets to the correlation. Thus, during operation, the process can continue to maintain and update the correlation with data sets including wash cycle times and the ICUMSA/total phenolics of the raw sugar.

### Example 2—Effect of Polyphenols on GI of Sugar

The effect of polyphenol content on the GI of sugar was studied. Traditional white sugar (ie essentially sucrose) was used as a control. Sugars with varied quantities of polyphenols were prepared by adding various amounts of polyphenol content to traditional white sugar.

Table 5 shows the results of testing of an in vitro Glycemic Index Speed Test (GIST) on the sugars prepared. The method involved in vitro digestion and analysis using Bruker BBFO 400 MHz NMR Spectroscopy. The testing was conducted by the Singapore Polytechnic Food Innovation & Resource Centre, who have demonstrated a strong correlation between the results of their in vitro method and traditional in vivo GI testing.

TABLE 5
sugar polyphenol content v GI

| | Polyphenol content (CE | | |
|---|---|---|---|
| Sample | polyphenols/100 g carbohydrates) | GI number | GI |
| 1 | 0 mg | About 68 | Medium |
| 2 | 30 mg | <55 (about 53) | Low |
| 3 | 60 mg | <20 (about 15) | Very Low |
| 4 | 120 mg | >68 (about 65) | Medium |

While the GI of fructose is 19, the GI of glucose is 100 out of 100. We therefore expect that as the glucose increases in less refined sugars the glycaemic response also concurrently increases.

A second set of sugars were prepared in which reducing sugars (1:1 glucose to fructose) were added to some of the white refined sugar plus polyphenol sugars. The GI of these sugars was also tested using the GIST method and the results are in Table 6.

TABLE 6
Effect of polyphenol and reducing sugar content on GI

| Sample # | Name of Material/Sample | Sample Code | GI Banding |
|---|---|---|---|
| 1 | Sugar + 30 PP + <0.16% RS | GI103 | Low |
| 2 | Sugar + 30 PP + 0.3% RS | GI104 | Medium |
| 3 | Sugar + 30 PP + 0.6% RS | GI105 | Medium/High |
| | | | (about 70) |
| 4 | Sugar + 60 PP + 0% RS | GI106 | Very low |
| | | | (about 15) |
| 5 | Sugar + 60 PP + 0.6% RS | GI107 | Low (about 29) |
| 6 | Sugar + 120 PP + 0% RS | GI108 | Med (about |
| | | | 65) |
| 7 | Sugar + 120 PP + 1.2% RS | GI109 | High (about |
| | | | 75) |

*PP = polyphenols in mg CE/100 g carbohydrates; RS = reducing sugars in % w/w (1:1 glucose:fructose)

The methods and systems described herein can be used in the production of a sugar product as described in the International Patent Publication No. WO2018018090 with the title “Sugar composition”.

It will be understood that the invention disclosed and defined in this specification extends to all alternative combinations of two or more of the individual features mentioned or evident from the text or drawings. All of these different combinations constitute various alternative aspects of the invention.

1. A method for producing a sugar product including:
receiving a first input in a control system representative of a pre-treatment sugar composition characteristic or an additive composition characteristic;
receiving a second input in the control system representative of a post-treatment sugar product target specification;
using the control system to determine at least one operating parameter for addition of an additive to the pre-treatment sugar and operating the addition of the additive in accordance with the at least one determined operating parameter, wherein the at least one determined operating parameter is determined from at least:
the first input,
the second input, and
a correlation relating at least the first input and the second input to the at least one operating parameter; and
treating the pre-treatment sugar composition by addition of the additive to produce a post-treatment sugar product with a characteristic that is at or nearer to the target specification than the characteristic of the pre-treatment sugar composition.

2. The method of claim 1, wherein the correlation is derived from a database of historical first inputs and corresponding historical output characterisation data and associated operating parameter(s).

3. The method of claim 2, wherein after the step of subjecting the pre-treatment sugar composition to the addition of the additive, the process further includes:
obtaining corresponding output characterisation data from the post-treatment sugar product; and
updating the database with the first input, second input and/or third input, the corresponding output characterisation data, and the operating parameter(s).

4. The method of claim 1, wherein the pre-treatment sugar composition characteristic is a pre-treatment spectrum, the additive characteristic is an additive spectrum and the post-treatment sugar product target specification is a post-treatment spectrum.

5. The method of claim 4, wherein each spectrum is selected from the group consisting of: a colour spectrum, a near infrared (NIR) spectrum, and/or a UV-vis spectrum.

6. The method of claim 5, wherein each spectrum is an NIR spectrum.

7. The method of claim 4, wherein each spectrum is indicative of a property selected from the group consisting of: flavonoid types and/or concentrations, phenol types and/or concentrations, polyphenol types and/or concentrations, tannin types and/or concentrations, caramel compound types and/or concentrations, reducing sugar types and/or concentrations, moisture, pol, or grain size.

8. The method of claim 4, wherein each spectrum is indicative of tricin concentration.

9. The method of claim 1, wherein the additive is added to the pre-treatment sugar by a spray system.

10. The method of claim 9, wherein the operating parameter(s) includes determining an operating parameter(s) selected from the group consisting of: a spray time, or a spray volume.

11. The method of claim 1, wherein the additive is at most about 50% w/w carbohydrates or about 5% to about 30% w/w carbohydrates.

12. The method of claim 1, wherein the pre-treatment sugar is a refined white sugar.

13. The method of claim 1, wherein the pre-treatment sugar comprises 5 to 40 mg CE polyphenols/100 g carbohydrates.

14. The method of claim 1, wherein the post-treatment sugar is low glycaemic.

15. The method of claim 1, wherein the post-treatment sugar is very low glycaemic.

16. The method of claim 1, wherein the post-treatment sugar comprises at least about 80% w/w sucrose and about 46 mg CE polyphenols/100 g carbohydrates to about 100 mg CE polyphenols/100 g carbohydrates or about 37 mg GAE polyphenols/100 g carbohydrates to about 80 mg GAE polyphenols/100 g carbohydrates.

17. The method of claim 1, wherein the post-treatment sugar comprises about 15 mg CE polyphenols/100 g carbohydrates to about 45 mg CE polyphenols/100 g carbohydrates or about 12 mg GAE polyphenols/100 g carbohydrates to about 37 mg GAE polyphenols/100 g carbohydrates.

18-24. (canceled)

25. The method of claim 1, wherein the method further comprises receiving a first output in the control system representative of a post-treatment sugar product characteristic.

26. A method for producing a sugar product including:
receiving a first input in a control system representative of a pre-treatment sugar composition characteristic;
receiving a second input in the control system representative of a post-treatment sugar product target specification;
receiving a third input in the control system representative of an additive composition characteristic;
using the control system to determine at least one operating parameter for addition of an additive to the pre-treatment sugar and operating the addition of the additive in accordance with the at least one determined operating parameter, wherein the at least one determined operating parameter is determined from two or more of the inputs selected from:
the first input,
the second input,
the third input, and
a correlation relating at least two or more of the inputs selected from the first input, the second input and the third input to the at least one operating parameter; and
treating the pre-treatment sugar composition by addition of the additive to produce a post-treatment sugar product with a characteristic that is at or nearer to the target specification than the characteristic of the pre-treatment sugar composition.

27. The method of claim 1, wherein the at least one determined operating parameter is determined from all three of the inputs by a correlation relating all three of the inputs to the at least one operating parameter.
